(19) United States
(12) Patent Application Publication
(10) Pub. No.: US 2013/0295609 A1
(43) Pub. Date: Nov. 7, 2013

(54) PLANT EXPRESSION SYSTEM

(71) Applicants: Marc-Andre D'Aoust, Quebec (CA); Pierre-Olivier Lavoie, Quebec (CA); Louis-Philippe Vezina, Neuville (CA)

(72) Inventors: Marc-Andre D'Aoust, Quebec (CA); Pierre-Olivier Lavoie, Quebec (CA); Louis-Philippe Vezina, Neuville (CA)

(73) Assignee: MEDICAGO INC., Quebec, (CA)

(21) Appl. No.: 13/883,439

(22) PCT Filed: Nov. 3, 2011
(86) PCT No.: PCT/CA11/01228

§ 371 Date: Jul. 17, 2013

Related U.S. Application Data
(60) Provisional application No. 61/410,241, filed on Nov. 4, 2010.

(51) Int. Cl. C07K 14/005

(52) U.S. Cl. CPC C07K 14/005
USPC 435/69.3; 435/320.1

(57) ABSTRACT

A plant expression system and methods for expressing a protein of interest in a plant are provided. The plant expression system comprises a first nucleic acid sequence regulatory region sequence, operatively linked with a one or more than one comovirus enhancer, a nucleotide sequence of interest, one or more than one geminivirus amplification elements, and a second nucleic acid encoding a geminivirus replicase. The method of producing a protein of interest in a plant, involves introducing the plant expression system into a plant, or portion of the plant, and incubating the plant or the portion of the plant under conditions that permit the expression of the nucleotide sequence and producing the protein of interest.

### TECHNICAL FIELD

The present invention relates to the expression of proteins of interest in plants. The present invention also provides methods and compositions for the production of proteins of interest in plants.

### BACKGROUND

Influenza is the leading cause of death in humans due to a respiratory virus. Common symptoms include fever, sore throat, shortness of breath, and muscle soreness, among others. During flu season, influenza viruses infect 10-20% of the population worldwide, leading to 250-500,000 deaths annually. Influenza pandemics are usually caused by highly transmittable and virulent influenza viruses, and can lead to elevated levels of illness and death globally. The emergence of new influenza A subtypes resulted in 4 major pandemics in the 20th century resulting in significant mortality and economic disruption. There is increasing concern that the virus may become highly infectious for humans. The major problem for human health is the fact that influenza viruses are antigenically unstable, that is, they mutate rapidly.

Influenza viruses are classified into types A, B, or C, based on the nucleoproteins and matrix protein antigens present. Influenza type A viruses may be further divided into subtypes according to the combination of hemagglutinin (HA) and neuraminidase (NA) surface glycoproteins presented. HA governs the ability of the virus to bind to and penetrate the host cell. NA removes terminal sialic acid residues from glycan chains on host cell and viral surface proteins, which prevents viral aggregation and facilitates virus mobility.

The current method of combating influenza in humans is by annual vaccination. Each year, the World Health Organization selects certain viral strains for inclusion in the annual influenza vaccine, which is produced in fertilized eggs. However, the number of vaccine doses produced each year is not sufficient to vaccinate the world's population. Furthermore, this production method faces a number of possible drawbacks including the possibility of contamination due to the use of whole virus, variable yields depending on the virus strain, extensive planning requirements for obtaining eggs, contamination risks due to chemicals used in purification, and long production times. Moreover, persons hypersensitive to egg proteins may not be eligible for receiving vaccines produced via this method.

Alternative production methods have been investigated. For example, influenza virus has been produced in mammalian cell cultures of, for example, in MDCK or PERC.6 cells, or the like. Another approach is reverse genetics, in which viruses are produced by cell transformation with viral genes. These methods, however, also requires the use of whole virus as well as elaborate methods and specific culture environments.

In order to protect the world population from influenza and to stave off future pandemics, vaccine manufacturers will need to develop effective, rapid methods producing vaccine doses. The current use of fertilized eggs to produce vaccines is insufficient and involves a lengthy process. Recombinant technologies offer promising approaches to the production of influenza antigens. However, the production of hemagglutinin has been limited to membrane-associated protein, which involves complex extraction processes with low yields, or to poorly-immunogenic soluble proteins.

Plants offer great potential as production systems for recombinant proteins. One approach to producing foreign proteins in plants is to generate stable transgenic plant lines. However this is a time consuming and labor intensive process. An alternative to transgenic plants is the use of plant virus-based expression vectors. Plant virus-based vectors allow for the rapid, high level, transient expression of proteins in plants.

Many different plant viruses have been modified to function as expression vectors. For example, Cowpea Mosaic Virus (CPMV) has been utilized for producing a variety of proteins (see, for example, WO2007/135480; WO2009/087391; Sainsbury F. et al., 2008, Plant Physiology; 148: 121-1218; Sainsbury F. et al., 2008, Plant Biotechnology Journal; 6: 82-92; Sainsbury F. et al., 2009, Plant Biotechnology Journal; 7: 682-693).

A geminivirus-derived, plant-based system has been proposed for the production of virus-like particles (Huang et al., 2009, Biotechnology and Bioengineering, 103: 706-714). Co-delivery of bean yellow dwarf virus (BeYDV)-derived vector and Rep/RepA-supplying vector by agroinfiltration of Nicotiana benthamiana leaves resulted in replicon amplification and protein production.

Plant-based expression systems preferably have a number of properties such as, for example, containing convenient cloning sites for genes of interest, can easily infect plants in a cost-effective manner, and/or can cause efficient local/systemic infection of inoculated plants. In addition, the infection should provide a good yield of useful protein material. Despite the advances made in modifying viruses to serve as expression systems in plants there is still room for improvement.

### SUMMARY

The present invention relates to the expression of proteins of interest in plants. The present invention also provides methods and compositions for the production of proteins of interest in plants. Also provided are nucleic acids and expression systems to produce proteins of interest in plants. The expression systems comprise a comovirus-based expression cassette and a geminivirus-based amplification element. Plant cells, plant tissues, whole plants, nucleic acids comprising gene constructs encoding the protein of interest, and methods of expressing proteins of interest in plants are also provided.

The present disclosure also provides an expression system for expressing hemagglutinin protein in plants.

The present invention further provides a plant expression system (A) comprising a first nucleic acid sequence comprising a regulatory region, operatively linked with a one or more than one comovirus enhancer, a nucleotide sequence of interest, and one or more than one geminivirus amplification element, and a second nucleic acid encoding a geminivirus replicase. The regulatory region may be selected from a plastocyanin promoter, a CaMV 35S promoter, a 2× CaMV35S promoter, a CAS promoter, a RbcS promoter, a Ubi promoter, or an actin promoter. Furthermore the one or more than one comovirus enhancer may be a comovirus 5′ UTR, for example, a Cowpea Mosaic Virus (CPMV) 5′UTR, or the CPMV 5′UTR may comprise the nucleotide sequence of SEQ ID NO:23. Furthermore, the one or more than one comovirus enhancer may comprises a comovirus 3′ UTR, for example, a Cowpea Mosaic Virus 3′ UTR, and a plastocyanin 3′ UTR. In addition, the one or more than one geminivirus amplification element may be selected from a Bean Yellow Dwarf Virus long intergenic region (BeYDV LIR), and a BeYDV short intergenic region (BeYDV SIR).

The present invention also provides the plant expression system (A) as described above, wherein the first nucleic acid sequence and the second nucleic acid sequence may be located on the same nucleic acid molecule, or they may be located on different DNA molecules.

The plant expression system (A) as defined above may further comprise a third nucleic acid sequence, the third nucleic acid sequence encoding a suppressor of silencing. The suppressor of silencing may be selected from the group HcPro and p19. The third nucleic acid sequence and the first nucleic acid sequence may be located on the same molecule, or the third nucleic acid sequence and the first nucleic acid sequence may be located on different molecules. The plant expression system may also comprises a forth nucleic acid sequence, the forth nucleic acid sequence encoding a chaperone protein. The chaperone protein may be selected from the group Hsp40 and Hsp70. The forth nucleic acid sequence and the first nucleic acid sequence may be located on the same molecule, or the forth nucleic acid sequence and the first nucleic acid sequence may be located on different DNA molecules.

The present invention provides the plant expression system (A) as defined above, wherein the nucleotide sequence of interest encodes an influenza hemagglutinin. The influenza hemagglutinin may comprises a native signal peptide sequence, or a non-native signal peptide. Furthermore, the influenza hemagglutinin may be selected from the group consisting of H1, H2, H3, H4, H5, H6, H7, H8, H9, H10, H11, H12, H13, H14, H15, and H16. The influenza hemagglutinin may also be selected from the group consisting of influenza type B hemagglutinin, influenza type A subtype H1 hemagglutinin, influenza type A subtype H3 hemagglutinin, and influenza type A subtype H5 hemagglutinin.

The present invention also pertains to a plant expression system (B) comprising a nucleic acid sequence comprising a regulatory sequence operatively linked to one or more than one comovirus enhancer; a nucleotide sequence of interest; one or more than one geminivirus amplification element, and a nucleic acid sequence encoding a geminivirus replicase protein. The regulatory region may be selected from a plastocyanin promoter, a CaMV 35S promoter, a 2× CaMV35S promoter, a CAS promoter, a RbcS promoter, a Ubi promoter, or an actin promoter. Furthermore the one or more than one comovirus enhancer may be a comovirus 5′ UTR, for example, a Cowpea Mosaic Virus (CPMV) 5′UTR, or the CPMV 5′UTR may comprise the nucleotide sequence of SEQ ID NO:23. Furthermore, the one or more than one comovirus enhancer may comprises a comovirus 3′ UTR, for example, a Cowpea Mosaic Virus 3′ UTR, and a plastocyanin 3′ UTR. In addition, the one or more than one geminivirus amplification element may be selected from a Bean Yellow Dwarf Virus long intergenic region (BeYDV LIR), and a BeYDV short intergenic region (BeYDV SIR).

The plant expression system (B) as defined above may further comprise a second nucleic acid sequence, the second nucleic acid sequence encoding a suppressor of silencing. The suppressor of silencing may be selected from the group HcPro and p19. The second nucleic acid sequence and the nucleic acid sequence (comprising the regulatory sequence operatively linked to one or more than one comovirus enhancer; the nucleotide sequence of interest; one or more than one geminivirus amplification element, and the nucleic acid sequence encoding a geminivirus replicase protein) may be located on the same molecule, or the second nucleic acid sequence and the nucleic acid sequence may be located on different molecules. The plant expression system may also comprises a third nucleic acid sequence, the third nucleic acid sequence encoding a chaperone protein. The chaperone protein may be selected from the group Hsp40 and Hsp70. The third nucleic acid sequence and the nucleic acid sequence (comprising the regulatory sequence operatively linked to one or more than one comovirus enhancer; the nucleotide sequence of interest; one or more than one geminivirus amplification element, and the nucleic acid sequence encoding a geminivirus replicase protein) may be located on the same molecule, or the third nucleic acid sequence and the nucleic acid sequence may be located on different DNA molecules.

The present invention provides the plant expression system (B) as defined above, wherein the nucleotide sequence of interest encodes an influenza hemagglutinin. The influenza hemagglutinin may comprises a native signal peptide sequence, or a non-native signal peptide. Furthermore, the influenza hemagglutinin may be selected from the group consisting of H1, H2, H3, H4, H5, H6, H7, H8, H9, H10, H11, H12, H13, H14, H15, and H16. The influenza hemagglutinin may also be selected from the group consisting of influenza type B hemagglutinin, influenza type A subtype H1 hemagglutinin, influenza type A subtype H3 hemagglutinin, and influenza type A subtype H5 hemagglutinin.

The present invention also provides a method (A) of producing an influenza virus like particle (VLP) in a plant or in a portion of a plant, the method comprising,
introducing into the plant or in the portion of a plant the plant expression system comprising a first nucleic acid sequence comprising a regulatory region, operatively linked with a one or more than one comovirus enhancer, a nucleotide sequence of interest, the nucleotide sequence of interest encoding an influenza hemagglutinin, and one or more than one geminivirus amplification element,
a second nucleic acid encoding a geminivirus replicase, and
incubating the plant or the portion of a plant under conditions that permit expression of the nucleotide sequence encoding the influenza hemagglutinin, thereby producing the influenza VLP.

The present invention also pertains to the method (A) as described above, wherein in the step of introducing, the first nucleic acid sequence and the second nucleic acid sequence may be located on the same molecule, or the first nucleic acid sequence and the second nucleic acid sequence maybe located on different molecules. If the first nucleic acid sequence and the second nucleic acid sequence are located on different molecules, then the different molecules may be introduced in the plant or in the portion of a plant at the same time or the different molecules may be introduced in the plant or in the portion of a plant at different times, for example by successive infiltration.

The present invention also provides the method (A) as described above, wherein in the step of incubating, the expression of the nucleotide sequence encoding the influenza hemagglutinin is a transient expression. Furthermore, the method may include a step of harvesting the plant or the portion of a plant, thereby obtaining a harvested plant or portion of a plant containing the influenza VLP. The method may further comprising a step of isolating the influenza VLP from the harvested plant or portion of a plant containing the influenza VLP, thereby obtaining an isolated influenza VLP.

The present invention provides the method (A) as descried above, wherein in the step of introducing, the plant expression system further comprises a third nucleic acid sequence, the third nucleic acid sequence encoding a suppressor of silencing expression. The third nucleic acid sequence and the first nucleic acid sequence may be located on the same molecule, or the third nucleic acid sequence and the first nucleic acid sequence may be located on different molecules. If the third nucleic acid sequence and the first nucleic acid sequence are located on different molecules, then the different molecules may be introduced in the plant or in the portion of a plant at the same time. Furthermore, within the step of introducing, the plant expression system may further comprises a fourth nucleic acid sequence, the forth nucleic acid sequence encoding a chaperone protein. The fourth nucleic acid sequence and the first nucleic acid sequence may be located on the same molecule, or the fourth nucleic acid sequence and the first nucleic acid sequence may be located on different molecules. If the fourth nucleic acid sequence and the first nucleic acid sequence are located on different molecules, then the different molecules may be introduced in the plant or in the portion of a plant at the same time.

The present invention also provides a method (B) of producing a protein of interest in a plant or in a portion of a plant, the method comprising,
introducing in the plant or in the portion of a plant the plant expression system comprising a first nucleic acid sequence comprising a regulatory region, operatively linked with a one or more than one comovirus enhancer, a nucleotide sequence of interest, and one or more than one geminivirus amplification element, and a second nucleic acid encoding a geminivirus replicase, and
incubating the plant or the portion of a plant under conditions that permit expression of the nucleotide sequence encoding the protein of interest, thereby producing the protein of interest.

The present invention also pertains to the method (B) as described above, wherein in the step of introducing, the first nucleic acid sequence and the second nucleic acid sequence may be located on the same molecule, or the first nucleic acid sequence and the second nucleic acid sequence maybe located on different molecules. If the first nucleic acid sequence and the second nucleic acid sequence are located on different molecules, then the different molecules may be introduced in the plant or in the portion of a plant at the same time.

The present invention also provides the method (B) as described above, wherein in the step of incubating, the expression of the nucleotide sequence encoding the influenza hemagglutinin is a transient expression. Furthermore, the method may include a step of harvesting the plant or the portion of a plant, thereby obtaining a harvested plant or portion of a plant containing the protein of interest. The method may further comprising a step of isolating the protein of interest from the harvested plant or portion of a plant containing the protein of interest, thereby obtaining an isolated protein of interest.

The present invention provides the method (B) as descried above, wherein in the step of introducing, the plant expression system further comprises a third nucleic acid sequence, the third nucleic acid sequence encoding a suppressor of silencing expression. The third nucleic acid sequence and the first nucleic acid sequence may be located on the same molecule, or the third nucleic acid sequence and the first nucleic acid sequence may be located on different molecules. If the third nucleic acid sequence and the first nucleic acid sequence are located on different molecules, then the different molecules may be introduced in the plant or in the portion of a plant at the same time. Furthermore, within the step of introducing, the plant expression system may further comprises a fourth nucleic acid sequence, the forth nucleic acid sequence encoding a chaperone protein. The fourth nucleic acid sequence and the first nucleic acid sequence may be located on the same molecule, or the fourth nucleic acid sequence and the first nucleic acid sequence may be located on different molecules. If the fourth nucleic acid sequence and the first nucleic acid sequence are located on different molecules, then the different molecules may be introduced in the plant or in the portion of a plant at the same time.

This summary does not necessarily describe all features of the invention. Other aspects, features and advantages of the invention will be apparent to those of ordinary skill in the art upon review of the following description of specific embodiments of the invention.

### BRIEF DESCRIPTION OF THE DRAWINGS

These and other features of the invention will become more apparent from the following description in which reference is made to the appended drawings wherein:

FIG. 1 shows nucleotide sequences of several constructs described in the present invention. FIG. 1A shows the nucleotide sequence of expression cassette 972 (SEQ ID NO:1) comprising a sequence from PacI site (bold; upstream of the promoter) to AscI site (bold immediately downstream of the NOS terminator), and includes wild type HA0 of H5 A/Indonesia/5/2005 (underlined). FIG. 1B shows the nucleotide sequence of expression cassette 560 (SEQ ID NO:2) comprising a sequence from PacI site (bold; upstream of the promoter) to AscI site (bold; immediately downstream of the NOS terminator). PDI signal peptide (PDISP)-HA0 of H1 A/California/4/2009 (underlined). FIG. 1C shows the nucleotide sequence of expression cassette 971 (SEQ ID NO:3), comprising a sequence from PacI site (bold; upstream of the promoter) to AscI site (bold; immediately downstream of the NOS terminator). PDISP-HA0 of H3 A/Brisbane/10/2007 (underlined). FIG. 1D shows the nucleotide sequence of expression cassette 973 (SEQ ID NO:4), comprising a sequence from Pad site (bold; upstream of the promoter) to AscI site (bold; immediately downstream of the NOS terminator). PDISP-HA0 of B/Florida/4/2006 (underlined). FIG. 1E shows the nucleotide sequence of LIR-MCS-SIR+LIR (SEQ ID NO:9). LIR (long intergenic region)−MCS (Multiple cloning site)−short intergenic region (SIR)+LIR sequence of 849 construct. FIG. 1F shows the nucleotide sequence of expression cassette 853 (SEQ ID NO:10), comprising a sequence from AscI site (bold; upstream of the LIR) to PmeI site (bold; downstream of the SIR+LIR). PDISP-HA0 of B/Florida/4/2006 (underlined). FIG. 1G shows the nucleotide sequence of expression cassette 561 (SEQ ID NO:11) comprising a sequence from AscI site (bold; upstream of the LIR) to PmeI site (bold; downstream of the SIR+LIR). PDISP-HA0 of H1 A/California/4/2009 (underlined). FIG. 1H shows the nucleotide sequence of expression cassette 851 (SEQ ID NO:12) comprising a sequence from AscI site (upstream of the LIR) to PmeI site (downstream of the SIR+LIR). PDISP-HA0 of H3 A/Brisbane/10/2007 (underlined). FIG. 1I shows the nucleotide sequence of expression cassette 852 (SEQ ID NO:13) comprising a sequence from AscI site (upstream of the LIR) to PmeI site (downstream of the SIR+LIR). Native Sp with HA0 from H5 A/Indonesia/05/2005 (underlined). FIG. 1J shows the nucleotide sequence (SEQ ID NO:14) of LIR-MCS-SIR-Replicase-LIR sequence of 848 construct. FIG. 1K shows the nucleotide sequence of expression cassette 475 (SEQ ID NO:15) comprising nucleotides from AscI site (bold; upstream of the LIR) to PmeI site (bold; downstream of the SIR-C1/C2-LIR). PDISP-HA0 of B/Florida/4/2006 (underlined). FIG. 1L shows the nucleotide sequence of expression cassette 471 (SEQ ID NO:16) comprising nucleotides from Asci site (bold; upstream of the LIR) to PmeI site (bold; downstream of the SIR-C1/C2-LIR). PDISP-HA0 of HI A/California/4/2009 (underlined). FIG. 1M shows the nucleotide sequence of expression cassette 473 (SEQ ID NO:17) comprising nucleotides from AscI site (bold; upstream of the LIR) to PmeI site (bold; downstream of the SIR-C1/C2-LIR). PDISP-HA0 of H3 A/Brisbane/10/2007 (underlined). FIG. 1N shows the nucleotide sequence of expression cassette 474 (SEQ ID NO:18) from AscI site (bold; upstream of the LIR) to PmeI site (bold; downstream of the SIR-C1/C2-LIR). Native Sp with HA0 from H5 A/Indonesia/05/2005 (underlined).

FIG. 2 shows schematic representation of several constructs that can be used for transient expression of HA in Nicotiana benthamiana. FIG. 2A shows a schematic construct for CPMV-HT-based constructs for HA expression (constructs number 971, 972, 973 and 560). FIG. 2B shows a schematic construct CPMV-HT+BeYDV-based construct for HA expression (constructs number 851, 852, 853 and 561). FIG. 2C shows a schematic construct for BeYDV replicase expression construct (construct number 834). FIG. 2D shows a schematic construct for CPMV-HT+BeYDV-based construct comprising the replicase gene (C1/C2) under the control of LIR for HA expression (constructs number 471, 473, 474 and 475).

FIG. 3 shows a comparison of transient HA accumulation using CPMV-HT-based expression system alone or a CPMV-HT-based expression system in conjunction with the BeYDV amplification system. FIG. 3A shows a comparison of HAB (from strain B/Florida/4/2006) accumulation in plants using a CPMV-HT-based expression cassette (construct number 973; see FIGS. 1D and 2A), or in conjunction with a BeYDV amplification system (constructs number 853 and 834 (providing expression of viral replicase); see FIGS. 1F and 2B). Three plants were analyzed for each construct (1, 2 and 3). Twenty micrograms of proteins were loaded for each extract analyzed. FIG. 3B shows a comparison of H3 (from strain A/Brisbane/10/2007) accumulation in plants using a CPMV-HT-based expression cassette alone (construct number 971; see FIGS. 1C and 2A) or in conjunction with a BeYDV amplification system (construct number 851; see FIGS. 1H and 2B). Three plants were analyzed for each construct (1, 2 and 3).Twenty micrograms or five micrograms of proteins from transformed plants were loaded as indicated. Fifteen micrograms of proteins from non-transformed plants were added to the 5 μg loads. FIG. 3C shows a comparison of H5 (from strain A/Indonesia/05/05) accumulation in plants using a CPMV-HT-based expression cassette alone (construct number 972; see FIG. 1A and FIG. 2A) or in conjunction with a BeYDV amplification system (construct number 852 see FIGS. 1I and 2B). Leaves of three plants were pooled prior to extraction. The amount of proteins from transformed plants is indicated for each lane. To ensure equal loading, the extracts from transformed plants were completed to 4 μg with protein extract from non-transformed plants. FIG. 3D shows a comparison of H1 (from strain A/California/04/2009) accumulation in plants using a CPMV-HT-based expression cassette alone (construct number 560; see FIGS. 1B and 2A) or in conjunction with a BeYDV amplification system (construct number 561; see FIGS. 1G and 2B). For each construct, three plants were harvested on days 2, 3, 4 and 5 post infiltration (respectively indicated as d2, d3, d4 and d5). Leaves of three plants were pooled prior to extraction. Five micrograms of proteins from transformed plants were mixed with 15 μg of proteins from non-transformed plants prior to loading.

FIG. 4 shows a comparison of transient HA accumulation in plants using several CPMV-HT-based expression cassettes in conjunction with a BeYDV amplification system and a replicase provided on a separate plasmid, or on the same plasmid under the control of the LIR. FIG. 4A shows a comparison of HA (from strain B/Florida/4/2006) accumulation in plants using the CPMV-HT-based expression cassette in conjunction with a BeYDV amplification system (construct number 853; see FIGS. 1F and 2B; and Plastocyanin-P19; construct number 472) with a replicase gene on a separate plasmid, or on the same plasmid under the control of the LIR (construct number 475; see FIGS. 1K and 2D; and Plastocyanin-P19; construct number 472). Three plants were analyzed for each construct (1, 2 and 3). Twenty micrograms of proteins were loaded for each extract analyzed. FIG. 4B shows a comparison of H3 (from strain A/Brisbane/10/2007) accumulation in plants using the CPMV-HT-based expression cassette in conjunction with the BeYDV amplification system (construct number 851; see FIGS. 1H and 2B; and Plastocyanin-PI 9; construct number 472) with the replicase gene on a separate plasmid, or on the same plasmid under the control of the LIR (construct number 473; see FIGS. 1M and 2D; and Plastocyanin-P19; construct number 472). Three plants were analyzed for each construct (1, 2 and 3). Half a microgram of proteins from transformed plants was mixed with 10 μg of proteins from non-transformed plants prior to loading. FIG. 4C shows a comparison of H5 (from strain A/Indonesia/05/05) accumulation in plants using the CPMV-HT-based expression cassette in conjunction with the BeYDV amplification system (construct number 852; see FIGS. 1I and 2B; and Plastocyanin-P19; construct number 472) with the replicase gene on a separate plasmid, or on the same plasmid under the control of the LIR (construct number 474; see FIGS. 1N and 2D; and Plastocyanin-P19; construct number 472). Three plants were analyzed for each construct (1, 2 and 3). Half a microgram of proteins from transformed plants was mixed with 10 μg of proteins from non-transformed plants prior to loading. FIG. 4D shows a comparison of H1 (from strain A/California/04/2009) accumulation in plants using the CPMV-HT-based expression cassette in conjunction with the BeYDV amplification system (construct number 561; see FIGS. 1G and 2B; and Plastocyanin-P19; construct number 472) with the replicase gene on a separate plasmid, or on the same plasmid under the control of the LIR (construct number 471; see FIGS. 1L and 2D; and Plastocyanin-P19; construct number 472). Three plants were analyzed for each construct (1, 2 and 3). Two and a half micrograms of proteins from transformed plants were mixed with 10 μg of proteins from non-transformed plants prior to loading.

### DETAILED DESCRIPTION

The present invention relates to nucleic acids and expression systems to produce proteins of interest in plants. The expression systems comprise a comovirus-based expression cassette and a geminivirus-based amplification element. Plant cells, plant tissues, whole plants, inoculum, nucleic acids (e.g. gene constructs), comprising the proteins of interest, and methods of expressing protein of interest in plants are also provided.

The present invention further provides a plant expression system comprising a first nucleic acid sequence comprising a regulatory region, operatively linked with a one or more than one comovirus enhancer, a nucleotide sequence of interest, and one or more than one geminivirus amplification element, and a second nucleic acid encoding a geminivirus replicase. The first nucleic acid sequence and the second nucleic acid sequence may be located on the same nucleic acid molecule, or they may be located on different DNA molecules. For example, the plant expression system may comprise a nucleic acid sequence comprising a regulatory sequence operatively linked to one or more than one comovirus enhancer; a nucleotide sequence of interest; one or more than one geminivirus amplification element, and a nucleic acid sequence encoding a geminivirus replicase protein.

The present invention also provides a nucleic acid comprising a promoter (regulatory region) sequence, a comovirus regulatory region, one or more sequences encoding one or more proteins of interest and a geminivirus amplification element, provided on the same or a different nucleic acid. The nucleic acid may further comprising a sequence encoding a geminivirus replicase, a comovirus 3′ untranlsated region (UTR) or a plastocyanin 3′ UTR, or a combination of a geminivirus replicase and a comovirus 3′ UTR or a plastocyanin 3′ UTR.

In the description that follows, a number of terms are used extensively, the following definitions are provided to facilitate understanding of various aspects of the invention. Use of examples in the specification, including examples of terms, is for illustrative purposes only and is not intended to limit the scope and meaning of the embodiments of the invention herein.

The terms “polypeptide”, peptide” and “protein” are used interchangeably herein to refer to a polymer of amino acid residues. The terms apply to naturally occurring amino acid polymers as well as amino acid polymers in which one or more amino acid residues is a non-naturally occurring amino acid, e.g., an amino acid analog. As used herein, the terms encompass amino acid chains of any length, including full length proteins for example antigens, wherein the amino acid residues are linked by covalent peptide bonds.

As used herein, the term “derivative” in the context of a polypeptide or protein, e.g. an antibody, refers to a polypeptide or protein that comprises an amino acid sequence which has been altered by the introduction of amino acid residue substitutions, deletions or additions. The term “derivative” as used herein also refers to a polypeptide or protein which has been modified, i.e., by the covalent attachment of any type of molecule to the antibody. For example, a polypeptide or protein may be modified, e.g., by glycosylation, acetylation, pegylation, phosphorylation, amidation, derivatization by known protecting/blocking groups, proteolytic cleavage, linkage to a cellular ligand or other protein, etc. Derivatives, polypeptides or proteins may be produced by chemical modifications using techniques known to those of skill in the art, including, but not limited to specific chemical cleavage, acetylation, formylation, metabolic synthesis of tunicamycin, etc. In some embodiments a derivative, a polypeptide or protein possesses a similar or identical function as the polypeptide or protein from which it was derived.

The expression system as described herein comprises an expression cassette based on a bipartite virus, or virus with a bipartite genome. For example, the present bipartite viruses may be of the Comoviridae family. Genera of the Comoviridae family include Comovirus, Nepovirus, Fabavirus, Cheravirus and Sadwavirus. Comoviruses include Cowpea mosaic virus (CPMV), Cowpea severe mosaic virus (CPSMV), Squash mosaic virus (SqMV), Red clover mottle virus (RCMV), Bean pod mottle virus (BPMV), Turnip ringspot virus (TuRSV), Broad bean true mosaic virus (BBtMV), Broad bean stain virus (BBSV), Radish mosaic virus (RaMV). Examples of comoviruse RNA-2 sequences comprising enhancer elements that may be useful for various aspects of the invention include, but are not limited to: CPMV RNA-2 (GenBank Accession No. NC 003550), RCMV RNA-2 (GenBank Accession No. NC_{—}003738), BPMV RNA-2 (GenBank Accession No. NC_{—}003495), CPSMV RNA-2 (GenBank Accession No. NC_{—}003544), SqMV RNA-2 (GenBank Accession No. NC_{—}003800), TuRSV RNA-2 (GenBank Accession No. NC_{—}013219.1). BBtMV RNA-2 (GenBank Accession No. GU810904), BBSV RNA2 (GenBank Accession No. FJ028650), RaMV (GenBank Accession No. NC_{—}003800)

Segments of the bipartite comoviral RNA genome are referred to as RNA-1 and RNA-2. RNA-1 encodes the proteins involved in replication while RNA-2 encodes the proteins necessary for cell-to-cell movement and the two capsid proteins. Any suitable comovirus-based cassette may be used including CPMV, CPSMV, SqMV, RCMV, or BPMV, for example, the expression cassette may be based on CPMV.

“Expression cassette” refers to a nucleotide sequence comprising a nucleic acid of interest under the control of, and operably (or operatively) linked to, an appropriate promoter or other regulatory elements for transcription of the nucleic acid of interest in a host cell.

It has been shown that transformation of Nicotiana benthamiana with full-length, replication-competent cDNA copies of both genomic RNAs of CPMV can result in a productive infection (Liu et al., 2004, Virology 323, 37-48, herein incorporated by reference). Examples of CPMV-based expression cassettes are described in WO2007/135480; WO2009/087391; and Sainsbury F. et al., (2008, Plant Physiology; 148: 1212-1218; Sainsbury F. et al., (2008, Plant Biotechnology Journal; 6: 82-92; Sainsbury F. et al., 2009, Plant Biotechnology Journal; 7: 682-693; which documents are herein incorporated by reference). As an example, which is not to be considered limiting, the untranslated regions (UTRs) obtained from the genomic RNA 2 of the cowpea mosaic virus (CMV) in which the two first translation initiation codons found in the 5′leader sequence have been deleted, may be used as described in WO 2009/087391. When combined to the CaMV 35S promoter and the nopaline synthase (NOS) terminator, the modified CPMV UTRs enhanced translation of the flanking coding region. The CPMV-based expression system was named CPMV-HT (hyperanslatable).

As described herein, an expression enhancer sequence, which sequence is derived from (or shares homology with) the RNA-2 genome segment of a bipartite RNA virus, such as a comovirus, in which a target initiation site has been mutated, may be used for expressing a nucleic acid sequence of interest. The present invention further provides processes for increasing the expression, or translational enhancing activity, of a sequence derived from an RNA-2 genome segment of a bipartite virus, which processes comprise mutating a target initiation site therein.

“Enhancer” sequences (or enhancer elements), include sequences derived from (or sharing homology with) the RNA-2 genome segment of a bipartite RNA virus, such as a comovirus, in which a target initiation site has been mutated. Such sequences can enhance downstream expression of a heterologous ORF to which they are attached. Without limitation, it is believed that such sequences when present in transcribed RNA, can enhance translation of a heterologous ORF to which they are attached.

A “target initiation site” as referred to herein and in the context of an enhancer sequence, is the initiation site (start codon) of a wild- type RNA-2 genome segment of a bipartite virus, for example a comovirus, from which the enhancer sequence in question is derived. The target initiation site serves as the initiation site for the production (translation) of the 105K protein, which is the longer of two carboxy coterminal proteins encoded by the wild-type RNA-2 genome segment. Production of the 105K protein, is initiated at the initiation site at position 161 in the wild-type CPMV RNA-2 genome segment. Thus, a target initiation site in an enhancer sequence derived from the CPMV RNA-2 genome segment may be the initiation site at position 161 in the wild-type CPMV RNA-2. Mutations around the start codon at position 161 may have the same, or similar, effect as mutating the start codon at position 161 itself by, for example, disrupting the context around the start codon may mean that the start codon is by-passed more frequently.

The enhancer sequence may comprise an indirectly mutated target initiation site produced by mutating one or more nucleotides upstream and/or downstream of the target initiation site, but retaining the wild-type target initiation site. The effect of mutating the upstream, downstream, or both an upstream and downstream nucleotides, is the same, or similar, to the effect observed when the target initiation site itself is mutated.

As target initiation sites serve as the initiation site for the production of the longer of two carboxy coterminal proteins encoded by a wild-type RNA-2 genome segment, it follows that target initiation sites are in-frame (in phase) with a second initiation site on the same wild-type RNA-2 genome segment, which serves as the initiation site for the production of the shorter of two carboxy coterminal proteins encoded by the wild-type RNA-2. Two initiation sites are in-frame if they are in the same triplet reading frame. For example, the target initiation site in an enhancer sequence derived from the wild-type CPMV RNA- 2 genome segment (the initiation site at position 161), is in frame with the initiation site at position 512, which serves as the initiation site for the production of the shorter of the two carboxy coterminal proteins encoded by CPMV RNA-2 (the 95K protein) in the wild-type CPMV RNA-2 genome segment.

Thus, a target initiation site that is located upstream (5′) of a second initiation site in the wild-type RNA-2 genome segment from which the enhancer sequence is derived, may serve as the initiation site for the production of the shorter of two carboxy coterminal polyproteins encoded by the wild-type RNA-2 genome segment. In addition, a target initiation site may also be located downstream (3′) of a third initiation site in the wild-type RNA-2 genome from which the enhancer sequence is derived. In CPMV, the target initiation site at position 161, is located upstream of a second initiation site at position 512 which serves as the initiation site for the production of the 95K protein. A third initiation site at position 115 is also present. A target initiation site in an enhancer sequence derived from the RNA-2 genome segment of a bipartite virus is therefore the first of two initiation sites for the production of two carboxy coterminal proteins encoded by the wild-type RNA-2. ‘First’ in this context refers to the initiation site located closer to the 5′ end of the wild-type RNA-2 genome segment.

More than one initiation site in the sequence may be mutated, if desired. For example the ‘third’ initiation site at (or corresponding to) position 115 may also be deleted or altered. It has been shown that removal of AUG 115 in addition to the removal of AUG 161 further enhances expression (Sainsbury and Lomonossoff, 2008, Plant Physiology; 148: 1212-1218). For example, the enhancer sequences of the present invention may be based on modified sequences from the RNA-2 genome segments of bipartite RNA viruses.

The sequences of the RNA-2 genome segments of these comoviruses and several specific strains are available from the NCBI database under the accession numbers listed in brackets: cowpea mosaic virus RNA-2 (NC_{—}003550), cowpea severe mosaic virus RNA-2 (NC_{—}003544), squash mosaic virus RNA-2 (NC_{—}003800), squash mosaic virus strain Kimble RNA-2 (AF059533), squash mosaic virus strain Arizona RNA-2 (AF059532), red clover mottle virus RNA-2 (NC_{—}003738), bean pod mottle virus RNA-2 (NC_{—}003495), bean pod mottle virus strain K-Hopkinsl RNA-2 (AF394609), bean pod mottle virus strain K-Hancock1 RNA-2 (AF394607), Andean potato mottle virus (APMoV: L16239) and Radish mosaic virus (RaMV; AB295644). There are also partial RNA-2 sequences available from bean rugose mosaic virus (BRMV; AF263548) and a tentative member of the genus Comovirus, turnip ringspot virus (EF191015). Numerous sequences from the other genera in the family Comoviridae are also available. To date, all comoviruses which have been investigated have been shown to have two alternative start codons for the expression of two carboxy coterminal polyproteins form their RNA-2 genome segments. In particular, the RNA-2 genome segments of CPMV, CPSMV, BPMV, SqMV and RCMV are known to comprise two alternative start codons for the expression of two carboxy coterminal polyproteins.

Target initiation sites in other comoviruses, which are equivalent to the initiation site at position 161 in the wild-type RNA-2 segment of CPMV can therefore be identified by methods as known in the art. For example, a sequence of interest may be aligned, using standard methods of sequence alignment such as BLAST, or by manual alignment, with that of CMPV to determine the location of a corresponding, equivalent initiation site.

As described herein, the enhancer sequence may comprise for example, nucleotides 1 to 512, or 1-509 of the CPMV RNA-2 genome segment, wherein the target initiation site at position 161 has been mutated. Alternatively, the enhancer sequence may comprise an equivalent sequence from another comovirus, wherein the target initiation site equivalent to the start codon at position 161 of CPMV has been mutated. The target initiation site may be mutated by substitution, deletion or insertion. For example, the target initiation site may be mutated by a point mutation. The CPMV enhancer may comprise SEQ ID NO: 23 as set forth below (mutated ATGs—ATG 115 and 161—are underlined):.

TATTAAAATCTTAATAGGTTTTGATAAAAGCGAACGTGGGGAAACCCGAA
CCAAACCTTCTTCTAAACTCTCTCTCATCTCTCTTAAAGCAAACTTCTCT
CTTGTCTTTCTTGCGTGAGCGATCTTCAACGTTGTCAGATCGTGCTTCGG
CACCAGTACAACGTTTTCTTTCACTGAAGCGAAATCAAAGATCTCTTTGT
GGACACGTAGTGCGGCGCCATTAAATAACGTGTACTTGTCCTATTCTTGT
CGGTGTGGTCTTGGGAAAAGAAAGCTTGCTGGAGGCTGCTGTTCAGCCCC
ATACATTACTTGTTACGATTCTGCTGACTTTCGGCGGGTGCAATATCTCT
ACTTCTGCTTGACGAGGTATTGTTGCCTGTACTTCTTTCTTCTTCTTCTT
GCTGATTGGTTCTATAAGAAATCTAGTATTTTCTTTGAAACAGAGTTTTC
CCGTGGTTTTCGAACTTGGAGAAAGATTGTTAAGCTTCTGTATATTCTGC
CCAAATTTG

The present enhancer sequence may have at least 99%, 98%, 97%, 96%, 95%, 90%, 85%, 80%, 75%, 70%, 65%, 60%, 55%, or 50% identity to the CPMV RNA-2 sequence, or any amount therebetween, wherein the target initiation site corresponding to position 161 of the wild-type CPMV RNA-2 genome segment has been mutated by substitution, deletion or insertion. For example the enhancer sequence may have from about 80% to about 99%, from about 90% to about 99%, or about 95% to about 99%, identity to the CPMV RNA-2 sequence, or any amount therebetween, wherein the target initiation site corresponding to position 161 of the wild-type CPMV RNA-2 genome segment has been mutated by substitution, deletion or insertion.

The terms “percent similarity”, “percent identity” and “percent homology” when referring to a particular sequence are used for example as set forth in the University of Wisconsin GCG software program. Methods of alignment of sequences for comparison are well-known in the art. Optimal alignment of sequences for comparison can be conducted, using for example the algorithm of Smith & Waterman, (1981, Adv. Appl. Math. 2:482), by the alignment algorithm of Needleman & Wunsch, (1970, J. Mol. Biol. 48:443), by the search for similarity method of Pearson & Lipman, (1988, Proc. Nat'l. Acad. Sci. USA 85:2444), by computerized implementations of these algorithms (for example: GAP, BESTFIT, FASTA, and TFASTA in the Wisconsin Genetics Software Package, Genetics Computer Group (GCG), 575 Science Dr., Madison, Wis.), or by manual alignment and visual inspection (see, e.g., Current Protocols in Molecular Biology, Ausubel et al., eds. 1995 supplement).

An example of an algorithm suitable for determining percent sequence identity and sequence similarity are the BLAST and BLAST 2.0 algorithms, which are described in Altschul et al., (1977, Nuc. Acids Res. 25:3389-3402) and Altschul et al., (1990, J. Mol. Biol. 215:403-410), respectively. BLAST and BLAST 2.0 are used, with the parameters described herein, to determine percent sequence identity for the nucleic acids and proteins of the invention. For example the BLASTN program (for nucleotide sequences) may use as defaults a wordlength (W) of 11, an expectation (E) of 10, M=5, N=−4 and a comparison of both strands. For amino acid sequences, the BLASTP program may use as defaults a wordlength of 3, and expectation (E) of 10, and the BLOSUM62 scoring matrix (see Henikoff & Henikoff, 1989, Proc. Natl. Acad. Sci. USA 89:10915) alignments (B) of 50, expectation (E) of 10, M=5, N=−4, and a comparison of both strands. Software for performing BLAST analyses is publicly available through the National Center for Biotechnology Information (see URL: ncbi.nlm.nih.gov/).

Enhancer sequences may also hybridise, under stringent conditions, with the complementary sequence of the CPMV RNA-2, with the proviso that the target initiation site corresponding to position 161 in the wild-type CPMV RNA-2 genome segment has been mutated.

Hybridization under stringent hybridization conditions is known in the art (see for example Current Protocols in Molecular Biology, Ausubel et al., eds. 1995 and supplements; Maniatis et al., in Molecular Cloning (A Laboratory Manual), Cold Spring Harbor Laboratory, 1982; Sambrook and Russell, in Molecular Cloning: A Laboratory Manual, 3^{rd} edition 2001; each of which is incorporated herein by reference). An example of one such stringent hybridization conditions may be about 16-20 hours hybridization in 4× SSC at 65° C., followed by washing in 0.1×SSC at 65° C. for an hour, or 2 washes in 0.1×SSC at 65° C. each for 20 or 30 minutes. Alternatively, an exemplary stringent hybridization condition could be overnight (16-20 hours) in 50% formamide, 4×SSC at 42° C., followed by washing in 0.1×SSC at 65° C. for an hour, or 2 washes in 0.1×SSC at 65° C. each for 20 or 30 minutes, or overnight (16-20 hours), or hybridization in Church aqueous phosphate buffer (7% SDS; 0.5M NaPO₄ buffer pH 7.2; 10 mM EDTA) at 65° C., with 2 washes either at 50° C. in 0.1×SSC, 0.1% SDS for 20 or 30 minutes each, or 2 washes at 65° C. in 2×SSC, 0.1% SDS for 20 or 30 minutes each.

A target initiation site in an enhancer sequence of the invention may be mutated by deletion, insertion or substitution, such that it no longer functions as a translation initiation site. For example, a point mutation may be made at the position of the target initiation site in the enhancer sequence. Alternatively, the target initiation site in the enhancer sequence may be deleted either partially or in its entirety. For example, a deletion spanning the target initiation site in the enhancer sequence may be made. Deletions spanning the initiation site may be about 5, 10, 15, 20, 25, 30, 35, 40, 45, or 50 nucleotides in length, when compared with the sequence of the wild-type RNA-2 genome segment from which the enhancer sequence is derived, with the proviso that the deletion span the initiation site.

Without wishing to be bound by theory, mutation of the start codon at position 161 in CPMV is thought to lead to inactivation of a translational suppressor, which results in enhanced initiation of translation from start codons located downstream of the inactivated translational suppressor. Thus, the enhancer sequence for use as described herein may be derived from an RNA-2 genome segment of a bipartite virus, wherein the enhancer sequence comprises an inactivated translational suppressor sequence.

A translational suppressor sequence may be a sequence in the wild-type RNA-2 genome segment of the bipartite virus (e.g. a comovirus) from which the enhancer sequence in question is derived, which comprises, or consists of, the initiation site for the production (translation) of the longer of two carboxy coterminal proteins encoded by the wild-type RNA-2 genome segment. Translational suppressor sequences in enhancer sequences derived from the CPMV RNA-2 genome segment, are sequences comprising, or consisting of, the target initiation site described above. Thus, translational suppressor sequences comprise, or consist of, a target initiation site as defined above, and may be inactivated by mutagenesis as described above.

The present disclosure provides an isolated nucleic acid comprising an expression enhancer sequence as described above. “Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

The nucleic acid may thus comprise a portion, or fragment, of the RNA-2 genome segment of the bipartite RNA virus from which the enhancer is derived. For example, the nucleic acid may not comprise at least a portion of the coding region of the RNA-2 genome segment from which it is derived. The coding region may be the region of the RNA-2 genome segment encoding the shorter of two carboxy coterminal proteins. The nucleic acid may comprise the portion of an RNA-2 genome segment of a bipartite virus extending from the 5′ end of the wild-type RNA-2 genome segment to the initiation site from which production (translation) of the shorter of two carboxy coterminal proteins encoded by the wild-type RNA-2 genome segment is initiated.

As described herein, there is provided one or more expression systems comprising an enhancer sequence as described above. The expression system may also comprise a nucleic acid of interest encoding a protein of interest inserted downstream of the enhancer sequence.

By “gene of interest”, “nucleotide (or nucleic acid) sequence of interest”, or “coding region of interest”, it is meant any gene, nucleotide sequence, or coding region (these terms are used interchangeably) that is to be expressed within a host organism, for example a plant, and may produce a protein of interest. Such a nucleotide sequence of interest may include, but is not limited to, a gene or coding region whose product is an industrial enzyme, a protein supplement, a nutraceutical, a value-added product, or a fragment thereof for feed, food, or both feed and food use. A nucleotide sequence, or coding region of interest may also include a gene that encodes a pharmaceutically active protein, for example growth factors, growth regulators, antibodies, antigens, and fragments thereof, or their derivatives useful for immunization or vaccination and the like. Such proteins include, but are not limited to, interleukins, for example one or more than one of IL-1 to IL-24, IL-26 and IL-27, cytokines, Erythropoietin (EPO), insulin, G-CSF, GM-CSF, hPG-CSF, M-CSF or combinations thereof, interferons, for example, interferon-alpha, interferon-beta, interferon-gama, blood clotting factors, for example, Factor VIII, Factor IX, or tPA hGH, receptors, receptor agonists, antibodies, neuropolypeptides, insulin, vaccines, growth factors for example but not limited to epidermal growth factor, keratinocyte growth factor, transformation growth factor, growth regulators, antigens, autoantigens, fragments thereof, or combinations thereof.

The protein of interest may also include an influenza hemagglutinin (HA; see WO 2009/009876, which is incorporated herein by reference). HA is a homotrimeric membrane type I glycoprotein, generally comprising a signal peptide, an HA1 domain, and an HA2 domain comprising a membrane-spanning anchor site at the C-terminus and a small cytoplasmic tail. Nucleotide sequences encoding HA are well known and are available (see, for example, the BioDefense and Public Health Database (now Influenza Research Database; Squires et al., 2008 Nucleic Acids Research 36:D497-D503) at URL:

biohealthbase.org/GSearch/home.do?decorator=Influenza; or the databases maintained by theNational Center for Biotechnology Information (see URL: ncbi.nlm.nih.gov), both of which are incorporated herein by reference).

An HA protein may be of a type A influenza, a type B influenza, or is a subtype of type A influenza HA selected from the group of H1, H2, H3, H4, H5, H6, H7, H8, H9, H10, H11, H12, H13, H14, H15, and H16. In some aspects of the invention, the HA may be from a type A influenza, selected from the group H1, H2, H3, H5, H6, H7 and H9. Fragments of the HAs listed above may also be considered a protein of interest. Furthermore, domains from an HA type or subtype listed above may be combined to produce chimeric HA's (see for example WO2009/076778 which is incorporated herein by reference)

Examples of subtypes comprising HA proteins include A/New Caledonia/20/99 (HIN1), A/Indonesia/5/2006 (H5N1), A/chicken/New York/1995, A/herring gull/DE/677/88 (H2N8), A/Texas/32/2003, A/mallard/MN/33/00, A/duck/Shanghai/1/2000, A/northern pintail/TX/828189/02, A/Turkey/Ontario/6118/68(H8N4), A/shoveler/Iran/G54/03, A/chicken/Germany/N/1949(H10N7), A/duck/England/56(H11N6), A/duck/Alberta/60/76(H12N5), A/Gull/Maryland/704/77(H13N6), A/Mallard/Gurjev/263/82, A/duck/Australia/341/83 (H15N8), A/black-headed gull/Sweden/5/99(H16N3), B/Lee/40, C/Johannesburg/66, A/PuertoRico/8/34 (H1N1), A/Brisbane/59/2007 (H1N1), A/Solomon Islands 3/2006 (H1N1), A/Brisbane 10/2007 (H3N2), A/Wisconsin/67/2005 (H3N2), B/Malaysia/2506/2004, B/Florida/4/2006, A/Singapore/1/57 (H2N2), A/Anhui/1/2005 (H5N1), A/Vietnam/1194/2004 (H5N1), A/Teal/HongKong/W312/97 (H6N1), A/Equine/Prague/56 (H7N7), A/HongKong/1073/99 (H9N2)).

The HA protein may be an H1, H2, H3, H5, H6, H7 or H9 subtype. For example, the H1 protein may be from the A/New Caledonia/20/99 (H1N1), A/PuertoRico/8/34 (H1N1), A/Brisbane/59/2007 (H1N1), A/Solomon Islands 3/2006 (HIN1), A/California/04/2009 (H1N1) or A/California/07/2009 (H1N1) strain. The H3 protein may also be from the A/Brisbane 10/2007 (H3N2), A/Wisconsin/67/2005 (H3N2) or A/Perth/16/2009 (H3N2) strain. In a further aspect of the invention, the H2 protein may be from the A/Singapore/1/57 (H2N2) strain. The H5 protein may be from the A/Anhui/1/2005 (H5N1), A/Vietnam/1194/2004 (H5N1), or A/Indonesia/5/2005 strain. In an aspect of the invention, the H6 protein may be from the A/Teal/HongKong/W312/97 (H6N1) strain. The H7 protein may be from the A/Equine/Prague/56 (H7N7) strain. In an aspect of the invention, the H9 protein is from the A/HongKong/1073/99 (H9N2) strain. In a further aspect of the invention, the HA protein may be from an influenza virus may be a type B virus, including B/Malaysia/2506/2004, B/Florida/4/2006 or B/Brisbane/60/08. Examples of amino acid sequences of the HA proteins from H1, H2, H3, H5, H6, H7, H9 or B subtypes include sequences as described in WO 2009/009876, WO 2009/076778, WO 2010/003225 (which are incorporated herein by reference). The influenza virus HA protein may be H5 Indonesia.

The HA may comprise a native, or a non-native signal peptide; the non-native signal peptide may be of plant origin. For example, the signal peptide may be a protein disulfide isomerase signal peptide. The native signal peptide may correspond to that of the hemagglutinin being expressed, or may correspond to a second hemagglutinin.

The present invention also provides nucleic acid molecules comprising sequences encoding an HA protein. The nucleic acid molecules may further comprise one or more regulatory regions operatively linked to the sequence encoding an HA protein. The nucleic acid molecules may comprise a sequence encoding an H1, H2, H3, H4, H5, H6, H7, H8, H9, H10, H11, H12, H13, H14, H15, H16 or HA from type B influenza. For example, the HA protein encoded by the nucleic acid molecule may be an H1, H2, H3, H5, H6, H7, H9 subtype an HA from type B. The H1 protein encoded by the nucleic acid molecule may be from the A/New Caledonia/20/99 (H1N1), A/PuertoRico/8/34 (H1N1), A/Brisbane/59/2007 (H1N1), A/Solomon Islands 3/2006 (H1N1), A/California/04/2009 (H1N1) or A/California/07/2009 (H1N1) strain. The H3 protein encoded by the nucleic acid molecule may be from the A/Brisbane 10/2007 (H3N2), A/Wisconsin/67/2005 (H3N2) or A/Perth/16/2009 (H3N2) strain. The H2 protein encoded by the nucleic acid molecule may be from the A/Singapore/1/57 (H2N2) strain. The H5 protein encoded by the nucleic acid molecule may also be from the A/Anhui/1/2005 (H5N1), A/Vietnam/1194/2004 (H5N1), or A/Indonesia/5/2005 strain. The H6 protein encoded by the nucleic acid molecule may be from the A/Teal/HongKong/W312/97 (H6N1) strain. The H7 protein encoded by the nucleic acid molecule may also be from the A/Equine/Prague/56 (H7N7) strain. Additionally, the H9 protein encoded by the nucleic acid molecule may be from the A/HongKong/1073/99 (H9N2) strain. The HA protein from B type encoded by the nucleic acid may be from the B/Florida/4/2006, B/Malaysia/2506/2004 or B/Brisbane/60/08 strain. Examples of sequences of nucleic acid molecules encoding such HA proteins from H1, H2, H3, H5, H6, H7, H9 subtypes or B type include sequences as described in WO 2009/009876, WO 2009/076778, WO 2010/003225 (which are incorporated herein by reference). The nucleic acid sequence may encode the influenza virus HA protein H5 Indonesia.

If the nucleic acid sequence of interest encodes a product that is directly or indirectly toxic to the plant, then such toxicity may be reduced by selectively expressing the nucleotide sequence of interest within a desired tissue or at a desired stage of plant development.

The coding region of interest or the nucleotide sequence of interest may be expressed in any suitable plant host which is either transformed or comprises the nucleotide sequences, or nucleic acid molecules, or genetic constructs, or vectors of the present invention. Examples of suitable hosts include, but are not limited to, Arabidopsis, agricultural crops including for example canola, Brassica spp., maize, Nicotiana spp., (tobacco) for example, Nicotiana benthamiana, alfalfa, potato, sweet potato (Ipomoea batatus), ginseng, pea, oat, rice, soybean, wheat, barley, sunflower, cotton, corn, rye (Secale cereale), sorghum (Sorghum bicolor, Sorghum vulgare), safflower (Carthamus tinctorius).

Therefore as described herein, there is provided an expression system comprising: (a) an enhancer sequence as described above; and (b) a nucleotide sequence encoding a protein of interest, wherein the nucleotide sequence is located downstream of the enhancer sequence. The nucleotide sequence and protein of interest may be a heterologous to, and not encoded by, the wild-type bipartite RNA virus from which the enhancer sequence is derived.

Expression systems may be used to express a protein of interest in a host organism. For example, the protein of interest may be heterologous to the host organism in question, and introduced into the cells in question using genetic engineering, transformation methods, or transformation followed by breeding. A heterologous nucleotide sequence in an organism may replace an endogenous equivalent nucleotide sequence that would normally perform the same or a similar function, or the inserted sequence may be additional to the endogenous nucleotide sequence. Persons skilled in the art will understand that expression of a nucleotide sequence of interest will require the presence of an initiation site (AUG) located upstream of the gene to be expressed. Such initiation sites may be provided either as part of an enhancer sequence or as part of a nucleotide sequence encoding a protein of interest.

By “regulatory region” “regulatory element” or “promoter” it is meant a portion of nucleic acid typically, but not always, upstream of the protein coding region of a gene, which may be comprised of either DNA or RNA, or both DNA and RNA. When a regulatory region is active, and in operative association, or operatively linked, with a gene of interest, this may result in expression of the gene of interest. A regulatory element may be capable of mediating organ specificity, or controlling developmental or temporal gene activation. A “regulatory region” includes promoter elements, core promoter elements exhibiting a basal promoter activity, elements that are inducible in response to an external stimulus, elements that mediate promoter activity such as negative regulatory elements or transcriptional enhancers. “Regulatory region”, as used herein, also includes elements that are active following transcription, for example, regulatory elements that modulate gene expression such as translational and transcriptional enhancers, translational and transcriptional repressors, upstream activating sequences, and mRNA instability determinants. Several of these latter elements may be located proximal to the coding region.

In the context of this disclosure, the term “regulatory element” or “regulatory region” typically refers to a sequence of DNA, usually, but not always, upstream (5′) to the coding sequence of a structural gene, which controls the expression of the coding region by providing the recognition for RNA polymerase and/or other factors required for transcription to start at a particular site. However, it is to be understood that other nucleotide sequences, located within introns, or 3′ of the sequence may also contribute to the regulation of expression of a coding region of interest. An example of a regulatory element that provides for the recognition for RNA polymerase or other transcriptional factors to ensure initiation at a particular site is a promoter element. Most, but not all, eukaryotic promoter elements contain a TATA box, a conserved nucleic acid sequence comprised of adenosine and thymidine nucleotide base pairs usually situated approximately 25 base pairs upstream of a transcriptional start site. A promoter element may comprise a basal promoter element, responsible for the initiation of transcription, as well as other regulatory elements (as listed above) that modify gene expression.

There are several types of regulatory regions, including those that are developmentally regulated, inducible or constitutive. A regulatory region that is developmentally regulated, or controls the differential expression of a gene under its control, is activated within certain organs or tissues of an organ at specific times during the development of that organ or tissue. However, some regulatory regions that are developmentally regulated may preferentially be active within certain organs or tissues at specific developmental stages, they may also be active in a developmentally regulated manner, or at a basal level in other organs or tissues within the plant as well. Examples of tissue-specific regulatory regions, for example see-specific a regulatory region, include the napin promoter, and the cruciferin promoter (Rask et al., 1998, J. Plant Physiol. 152: 595-599; Bilodeau et al., 1994, Plant Cell 14: 125-130). An example of a leaf-specific promoter includes the plastocyanin promoter (see U.S. Pat. No. 7,125,978, which is incorporated herein by reference).

An inducible regulatory region is one that is capable of directly or indirectly activating transcription of one or more DNA sequences or genes in response to an inducer. In the absence of an inducer the DNA sequences or genes will not be transcribed. Typically the protein factor that binds specifically to an inducible regulatory region to activate transcription may be present in an inactive form, which is then directly or indirectly converted to the active form by the inducer. However, the protein factor may also be absent. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. A plant cell containing an inducible regulatory region may be exposed to an inducer by externally applying the inducer to the cell or plant such as by spraying, watering, heating or similar methods. Inducible regulatory elements may be derived from either plant or non-plant genes (e.g. Gatz, C. and Lenk, I.R.P., 1998, Trends Plant Sci. 3, 352-358; which is incorporated by reference). Examples, of potential inducible promoters include, but not limited to, tetracycline-inducible promoter (Gatz, C.,1997, Ann. Rev. Plant Physiol. Plant Mol. Biol. 48, 89-108; which is incorporated by reference), steroid inducible promoter (Aoyama, T. and Chua, N. H.,1997, Plant J. 2, 397-404; which is incorporated by reference) and ethanol-inducible promoter (Salter, M. G., et al, 1998, Plant Journal 16, 127-132; Caddick, M. X., et a1,1998, Nature Biotech. 16, 177-180, which are incorporated by reference) cytokinin inducible IB6 and CKI1 genes (Brandstatter, I. and Kieber, J. J.,1998, Plant Cell 10, 1009-1019; Kakimoto, T., 1996, Science 274, 982-985; which are incorporated by reference) and the auxin inducible element, DR5 (Ulmasov, T., et al., 1997, Plant Cell 9, 1963-1971; which is incorporated by reference).

A constitutive regulatory region directs the expression of a gene throughout the various parts of a plant and continuously throughout plant development. Examples of known constitutive regulatory elements include promoters associated with the CaMV 35S transcript. (p35S; Odell et al., 1985, Nature, 313: 810-812), the rice actin 1 (Zhang et al, 1991, Plant Cell, 3: 1155-1165), actin 2 (An et al., 1996, Plant J., 10: 107-121), or tms 2 (U.S. Pat. No. 5,428,147, which is incorporated herein by reference), and triosephosphate isomerase 1 (Xu et. al., 1994, Plant Physiol. 106: 459-467) genes, the maize ubiquitin 1 gene (Cornejo et al, 1993, Plant Mol. Biol. 29: 637-646), the Arabidopsis ubiquitin 1 and 6 genes (Holtorf et al, 1995, Plant Mol. Biol. 29: 637-646), the tobacco translational initiation factor 4A gene (Mandel et al, 1995 Plant Mol. Biol. 29: 995-1004). the Cassava Vein Mosaic Virus promoter, pCAS, (Verdaguer et al., 1996); the promoter of the small subunit of ribulose biphosphate carboxylase, pRbcS: (Outchkourov et al., 2003), the pUbi (for monocots and dicots).

As described herein, promoters comprising enhancer sequences with demonstrated efficiency in leaf expression, have been found to be effective in transient expression. Without wishing to be bound by theory, attachment of upstream regulatory elements of a photosynthetic gene by attachment to the nuclear matrix may mediate strong expression. For example up to -784 from the translation start site of the pea plastocyanin gene may be used mediate strong reporter gene expression.

The term “constitutive” as used herein does not necessarily indicate that a gene under control of the constitutive regulatory region is expressed at the same level in all cell types, but that the gene is expressed in a wide range of cell types even though variation in abundance is often observed.

By “operatively linked” it is meant that the particular sequences, for example a regulatory element and a coding region of interest, interact either directly or indirectly to carry out an intended function, such as mediation or modulation of gene expression. The interaction of operatively linked sequences may, for example, be mediated by proteins that interact with the operatively linked sequences.

The one or more than one nucleotide sequence of the present invention may be expressed in any suitable plant host that is transformed by the nucleotide sequence, or constructs, or vectors of the present invention. Examples of suitable hosts include, but are not limited to, Arabidopsis, agricultural crops including for example canola, Brassica spp., maize, Nicotiana spp., (tobacco) for example, Nicotiana benthamiana, alfalfa, potato, sweet potato (Ipomoea batatus), ginseng, pea, oat, rice, soybean, wheat, barley, sunflower, cotton, corn, rye (Secale cereale), sorghum (Sorghum bicolor, Sorghum vulgare), safflower (Carthamus tinctorius).

The one or more constructs of the present invention can further comprise a 3′ untranslated region. A 3′ untranslated region refers to that portion of a gene comprising a DNA segment that contains a polyadenylation signal and any other regulatory signals capable of effecting mRNA processing or gene expression. The polyadenylation signal is usually characterized by effecting the addition of polyadenylic acid tracks to the 3′ end of the mRNA precursor. Polyadenylation signals are commonly recognized by the presence of homology to the canonical form 5′ AATAAA-3′ although variations are not uncommon. One or more of the chimeric genetic constructs of the present invention can also include further enhancers, either translation or transcription enhancers, as may be required. These enhancer regions are well known to persons skilled in the art, and can include the ATG initiation codon and adjacent sequences. The initiation codon must be in phase with the reading frame of the coding sequence to ensure translation of the entire sequence.

Non-limiting examples of suitable 3′ regions are the 3′ transcribed non-translated regions containing a polyadenylation signal of Agrobacterium tumor inducing (Ti) plasmid genes, such as the nopaline synthase (Nos gene) and plant genes such as the soybean storage protein genes, the small subunit of the ribulose-1,5-bisphosphate carboxylase gene (ssRUBISCO; U.S. Pat. No. 4,962,028; which is incorporated herein by reference), the promoter used in regulating plastocyanin expression (Pwee and Gray 1993; which is incorporated herein by reference). The termination (terminator) sequence may also be a termination sequence derived from the RNA-2 genome segment of a bipartite RNA virus, e.g. a comovirus. For example, the termination sequence may be derived from the same bipartite RNA virus from which the enhancer sequence is derived. The termination sequence may comprise a stop codon. Termination sequence may also be followed by polyadenylation signals.

Expression systems as described herein may be operably linked to promoter and terminator sequences. Thus, an expression systems may further comprise a termination sequence and the gene encoding a protein of interest may be located between the enhancer sequence and the termination sequence, i.e. downstream (3′) of the enhancer sequence and upstream (5′) of the termination sequence. Thus the disclosure further provides an expression cassette comprising: (i) a promoter, operably linked to (ii) an enhancer sequence as described above (iii) a nucleotide sequence of interest, and (iv) a terminator sequence.

Expression cassettes, expression constructs and expression systems of the invention may also comprise an untranslated region (UTR). The UTR may be located upstream of a terminator sequence present in the expression cassette (gene expression construct or gene expression system). Where the expression cassette (gene expression construct or gene expression system) comprises a nucleic acid encoding a protein of interest, the UTR may be located downstream of the nucleic acid of interest (3′UTR). Thus, the UTR may be located between a nucleic acid encoding a protein of interest and a terminator sequence. The UTR may be derived from a bipartite RNA virus, e.g. from the RNA-2 genome segment of a bipartite RNA virus. The UTR may be the 3′ UTR of the same RNA-2 genome segment from which the enhancer sequence present in the expression cassette (expression construct or gene expression system) is derived. Preferably, the UTR is the 3′ UTR of a comoviral RNA-2 genome segment, e.g. the 3′ UTR of the CPMV RNA-2 genome segment.

The expression constructs as described above may be present in a vector. The vector may comprise border sequences which permit the transfer and integration of the expression cassette into the genome of the organism or host. The construct may be a plant binary vector, for example a binary transformation vector based on pPZP (Hajdukiewicz, et al. 1994). Other example constructs include pBin19 (see Frisch, D. A., L. W. Harris-Haller, et al. 1995, Plant Molecular Biology 27: 405-409).

If desired, the constructs of this invention may be further manipulated to include selectable markers. However, this may not be required. Useful selectable markers include enzymes that provide for resistance to chemicals such as an antibiotic for example, gentamycin, hygromycin, kanamycin, or herbicides such as phosphinothrycin, glyphosate, chlorosulfuron, and the like. Similarly, enzymes providing for production of a compound identifiable by colour change such as GUS (beta-glucuronidase), or luminescence, such as luciferase or GFP, may be used.

An example of a vector that may be used as described herein is a pEAQ vector which permits direct cloning by use of a polylinker between the 5′ leader and 3′ UTRs of an expression cassette. This vector may also include a translational enhancer as described herein, positioned on a T-DNA which also contains a suppressor of gene silencing and NPTII. The polylinker may also encode one or two sets of 6 x Histidine residues to allow the inclusion of N- or C-terminal His-tags to the protein of interest to facilitate protein purification.

Post-transcriptional gene silencing (PTGS) may be involved in limiting expression of transgenes in plants, and co-expression of a suppressor of silencing from the potato virus Y (HcPro) may be used to counteract the specific degradation of transgene mRNAs (Brigneti et al., 1998, EMBO J. 17, 6739-6746, which is incorporated herein by reference). Alternate suppressors of silencing are well known in the art and may be used as described herein (Chiba et al., 2006, Virology 346:7-14; which is incorporated herein by reference), for example but not limited to, TEV-pl/HC-Pro (Tobacco etch virus-pl/HC-Pro), BYV-p21, p19 of Tomato bushy stunt virus (TBSV p19; the construction of p19 is described in described in WO 2010/0003225, which is incorporated herein by reference), capsid protein of Tomato crinkle virus (TCV-CP), 2b of Cucumber mosaic virus; CMV-2b), p25 of Potato virus X (PVX-p25), p11 of Potato virus M (PVM-p11), p11 of Potato virus S (PVS-p11), p16 of Blueberry scorch virus, (BScV-p16), p23 of Citrus tristeza virus (CTV-p23), p24 of Grapevine leafroll-associated virus-2, (GLRaV-2 p24), p10 of Grapevine virus A, (GVA-p10), p14 of Grapevine virus B (GVB-p14), p10 of Heracleum latent virus (HLV-p 10), or p16 of Garlic common latent virus (GCLV-p 16).

Therefore, one or more suppressors of silencing, for example, but not limited to, HcPro, TEV -pl/HC-Pro, BYV-p21, TBSV p19, TCV-CP, CMV-2b, PVX-p25, rgscam, B2 protein from FHV, the small coat protein of CPMV, and coat protein from TCV, PVM-pll, PVS-pll, BScV-p16, CTV-p23, GLRaV-2 p24, GBV-p14, HLV-p10, GCLV-p16, or GVA-p10 may be co-expressed along with the comovirus-based expression cassette, geminivirus-derived amplification element, and the nucleic acid sequence encoding the protein of interest to further ensure high levels of protein production within a plant.

The expression systems may also comprise amplification elements from a geminivirus for example, an amplification element from the bean yellow dwarf virus (BeYDV). BeYDV belongs to the Mastreviruses genus adapted to dicotyledonous plants. BeYDV is monopartite having a single-strand circular DNA genome and can replicate to very high copy numbers by a rolling circle mechanism. BeYDV-derived DNA replicon vector systems have been used for rapid high-yield protein production in plants.

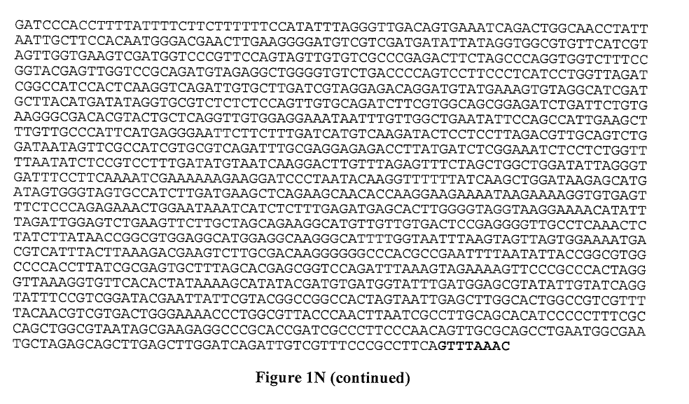

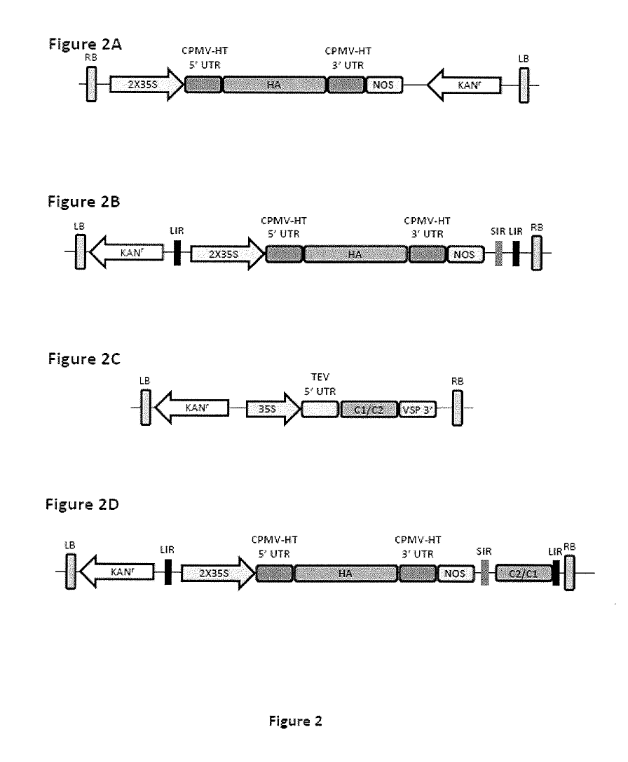

As used herein, the phrase “amplification elements” refers to a nucleic acid segment comprising at least a portion of one ore more long intergenic regions (or long intergenic repeat; LIR) of a geminivirus genome. As used herein, “long intergenic region”, “long intergenic repeat”, or “LIR”, refers to a region of a long intergenic region that contains a rep binding site capable of mediating excision and replication by a geminivirus Rep protein. In some aspects, the nucleic acid segment comprising one or more LIRs, may further comprises a short intergenic region (or small intergenic region; SIR) of a geminivirus genome. As used herein, “short intergenic region”, “small intergenic region” or “SIR”, refers to the complementary strand (the short IR (SIR) of a Mastreviruses). Any suitable geminivirus-derived amplification element may be used herein. See, for example, WO2000/20557; WO2010/025285; Zhang X. et al. (2005, Biotechnology and Bioengineering, Vol. 93, 271-279), Huang Z. et al. (2009, Biotechnology and Bioengineering, Vol. 103, 706-714), Huang Z. et al.(2009, Biotechnology and Bioengineering, Vol. 106, 9-17); which are herein incorporated by reference). As shown if FIGS. 2B and 2D, if more than one LIR is used in the construct, for example two LIRs, then the promoter, CMPV-HT regions and the nucleic acid sequence of interest and the terminator are bracketed by each of the two LIRs. Furthermore, the amplification element might for example originate from the sequence as disclosed in Halley-Stott et al. (2007) Archives of Virology 152: 1237-1240, deposited under Gen Bank accession number DQ458791, which are herein incorporated by reference. The nucleic acid segment comprising LIRs and SIRs may be determined by aligning the desired sequence with the sequences provided herein for BeYDV. For example, the nucleic acid segment comprising LIRs of the sequence, Gen Bank DQ458791, comprise nucleotides 2401 to 2566 and 1 to 128.The nucleic acid segment comprising SIRs are nucleotides 1154 to 1212.

As described herein, co-delivery of bean yellow dwarf virus (BeYDV)-derived vector and a Rep/RepA-supplying vector, by agroinfiltration of Nicotiana benthamiana leaves results in efficient replicon amplification and robust protein production.

A comovirus-based expression cassette and a geminivirus-derived amplification element may be comprised in respective, first and second vectors, or the component parts may be included in one vector. If two vectors are used, the first and second vectors may be introduced into a plant cell simultaneously or separately.

A viral replicase may also be included in the expression system as described herein to increase expression of the nucleic acid of interest. An non-limiting example of a replicase is a BeYDV replicase (pREP110) encoding BeYDV Rep and RepA (C2/C1; Huang et al., 2009, Biotechnol. Bioeng. 103, 706-714; which is incorporated herein by reference). Another non-limiting example of a replicase is disclosed in Halley-Stott et al. (2007, Archives of Virology 152: 1237-1240 which are herein incorporated by reference), and deposited under Gen Bank accession number DQ458791. The nucleic acid segment comprising C1:C2 gene comprises nucleotides 1310 to 2400.

By “co-expressed” it is meant that two or more than two nucleotide sequences are expressed at about the same time within the plant, and within the same tissue of the plant. However, the nucleotide sequences need not be expressed at exactly the same time. Rather, the two or more nucleotide sequences are expressed in a manner such that the encoded products have a chance to interact. The two or more than two nucleotide sequences can be co-expressed using a transient expression system, where the two or more sequences are introduced within the plant at about the same time under conditions that both sequences are expressed. Alternatively, a platform plant comprising one of the nucleotide sequences may be transformed in a stable manner, with an additional sequence encoding the protein of interest introduced into the platform plant in a transient manner.

The constructs of the present invention can be introduced into plant cells using Ti plasmids, Ri plasmids, plant virus vectors, direct DNA transformation, micro-injection, electroporation, etc. For reviews of such techniques see for example Weissbach and Weissbach, Methods for Plant Molecular Biology, Academy Press, New York VIII, pp. 421-463 (1988); Geierson and Corey, Plant Molecular Biology, 2d Ed. (1988); and Miki and Iyer, Fundamentals of Gene Transfer in Plants. In Plant Metabolism, 2d Ed. D T. Dennis, D H Turpin, D D Lefebrve, D B Layzell (eds), Addison Wesly, Langmans Ltd. London, pp. 561-579 (1997). Other methods include direct DNA uptake, the use of liposomes, electroporation, for example using protoplasts, micro-injection, microprojectiles or whiskers, and vacuum infiltration. See, for example, Bilang, et al. (1991, Gene 100: 247-250), Scheid et al. (1991, Mol. Gen. Genet. 228: 104-112), Guerche et al. (1987, Plant Science 52: 111-116), Neuhause et al. (1987, Theor. Appl Genet. 75: 30-36), Klein et al., (2987, Nature 327: 70-73); Freeman et al. (1984, Plant Cell Physiol. 29: 1353), Howell et al. (1980, Science 208: 1265), Horsch et al. (1985, Science 227: 1229-1231), DeBlock et al., (1989, Plant Physiology 91: 694-701), Methods for Plant Molecular Biology (Weissbach and Weissbach, eds., Academic Press Inc., 1988), Methods in Plant Molecular Biology (Schuler and Zielinski, eds., Academic Press Inc., 1989), WO 92/09696, WO 94/00583, EP 331083, EP 175966, Liu and Lomonossoff (2002, J Virol Meth, 105:343-348), EP 290395; WO 8706614; U.S. Pat. Nos. 4,945,050; 5,036,006; and 5,100,792, U.S. patent application Ser. Nos. 08/438,666, filed May 10, 1995, and Ser. No. 07/951,715, filed Sep. 25, 1992, (all of which are hereby incorporated by reference).

Transient expression methods may be used to express the constructs of the present invention (see D'Aoust et al., 2009, Methods in molecular biology, Vol 483, pages 41-50; Liu and Lomonossoff, 2002, Journal of Virological Methods, 105:343-348; which is incorporated herein by reference). Alternatively, a vacuum-based transient expression method, as described by Kapila et al., (1997, Plant Sci. 122, 101-108; which is incorporated herein by reference), or WO 00/063400, WO 00/037663 (which are incorporated herein by reference) may be used. These methods may include, for example, but are not limited to, a method of Agro-inoculation or Agro-infiltration, syringe infiltration, however, other transient methods may also be used as noted above. With Agro-inoculation, Agro-infiltration, or syringe infiltration, a mixture of Agrobacteria comprising the desired nucleic acid enter the intercellular spaces of a tissue, for example the leaves, aerial portion of the plant (including stem, leaves and flower), other portion of the plant (stem, root, flower), or the whole plant. After crossing the epidermis the Agrobacteria infect and transfer t-DNA copies into the cells. The t-DNA is episomally transcribed and the mRNA translated, leading to the production of the protein of interest in infected cells, however, the passage oft-DNA inside the nucleus is transient.

Also considered part of this invention are transgenic plants, plant cells or seeds containing the chimeric gene construct of the present invention that may be used as a platform plant suitable for transient protein expression described herein. Methods of regenerating whole plants from plant cells are also known in the art (for example see Guerineau and Mullineaux (1993, Plant transformation and expression vectors. In: Plant Molecular Biology Labfax (Croy RRD ed) Oxford, BIOS Scientific Publishers, pp 121-148). In general, transformed plant cells are cultured in an appropriate medium, which may contain selective agents such as antibiotics, where selectable markers are used to facilitate identification of transformed plant cells. Once callus forms, shoot formation can be encouraged by employing the appropriate plant hormones in accordance with known methods and the shoots transferred to rooting medium for regeneration of plants. The plants may then be used to establish repetitive generations, either from seeds or using vegetative propagation techniques. Transgenic plants can also be generated without using tissue culture. Methods for stable transformation, and regeneration of these organisms are established in the art and known to one of skill in the art. Available techniques are reviewed in Vasil et al., (Cell Culture and Somatic Cell Genetics of Plants, VoI I, II and III, Laboratory Procedures and Their Applications, Academic Press, 1984), and Weissbach and Weissbach, (Methods for Plant Molecular Biology, Academic Press, 1989). The method of obtaining transformed and regenerated plants is not critical to the present invention.

If plants, plant portion, or plant cells are to be transformed or co-transformed by two or more nucleic acid constructs, the nucleic acid construct may be introduced into the Agrobacterium in a single transfection event the nucleic acids are pooled, and the bacterial cells transfected as described. Alternately, the constructs may be introduced serially. In this case, a first construct is introduced to the Agrobacterium as described, the cells grown under selective conditions (e.g. in the presence of an antibiotic) where only the singly transformed bacteria can grow. Following this first selection step, a second nucleic acid construct is introduced to the Agrobacterum as described, and the cells grown under doubly-selective conditions, where only the doubly-transformed bacteria can grow. The doubly-transformed bacteria may then be used to transform a plant, plant portion or plant cell as described herein, or may be subjected to a further transformation step to accommodate a third nucleic acid construct.

The present disclosure further provides a transgenic plant comprising the present expression system, wherein the heterologous nucleic acid of interest in the cassette is expressed at an enhanced level when compared to other analogous expression systems that lack one or more components of the expression system as described herein.

The present disclosure further comprises a method for generating a protein of interest, comprising the steps of providing a plant, or plant part, that expresses the expression system as described herein, harvesting, at least, a tissue in which the protein of interest has been expressed and optionally, isolating the protein of interest from the tissue.

Thus in various aspects, and without limitation, the invention provides:
one or more expression systems comprising a comovirus-based expression cassette, one or more BeYDV amplification elements, a promoter, a nucleic acid encoding a protein of interest, or a polylinker, and optionally a terminator.
methods of expressing a protein of interest, in a host organism such as a plant using one or more expression systems or vectors as described herein.
host cells and organisms expressing proteins of interest from the one or more expression systems or vectors of the invention and methods of producing the hosts and organisms.

TABLE 1
lists of sequences

| SEQ ID NO: | Description | FIG. No |
|---|---|---|
| 1 | 972 - wild type HA0 of H5 | 1A |
| | A/Indonesia/5/2005 | |
| 2 | 560 - PDISP-HA0 of H1 | 1B |
| | A/California/4/2009 | |
| 3 | 971 - PDISP-HA0 of H3 | 1C |
| | A/Brisbane/10/2007 | |
| 4 | 973 - PDISP-HA0 of B/Florida/4/2006 | 1D |
| 5 | primer - MfeI-MluI-AscI-LIR.c | — |
| 6 | primer - SbfI-AflII-HindIII-LIR.r | — |
| 7 | primer - SacI-EcoRI-SIR.c | — |
| 8 | primer - SpeI-FseI-LIR.r | — |
| 9 | 849 - LIR-MCS-SIR + LIR | 1E |
| 10 | 853 - LIR-PDISP-HA0 of | 1F |
| | B/Florida/4/2006-SIR + LIR | |
| 11 | 561 - LIR-PDISP-HA0 of H1 | 1G |
| | A/California/4/2009-SIR + LIR | |
| 12 | 851 - LIR-PDISP-HA0 of H3 | 1H |
| | A/Brisbane/10/2007-SIR + LIR | |
| 13 | 852 - LIR-Native Sp with HA0 from H5 | 1I |
| | A/Indonesia/05/2005-SIR + LIR | |
| 14 | 848 - LIR-MCS-SIR-replicase-LIR | 1J |
| 15 | 475 - LIR-PDISP-HA0 of | 1K |
| | B/Florida/4/2006-SIR-C1/C2-LIR | |
| 16 | 471 - LIR-PDISP-HA0 of H1 | 1L |
| | A/California/4/2009-SIR-C1/C2-LIR | |
| 17 | 473 - LIR-PDISP-HA0 of H3 | 1M |
| | A/Brisbane/10/2007-SIR-C1/C2-LIR | |
| 18 | 474 - LIR-Native Sp with HA0 from H5 | 1N |
| | A/Indonesia/05/2005-SIR-C1/C2-LIR | |
| 19 | Primer - Plasto-443c | — |
| 20 | Primer - supP19-plasto.r | — |
| 21 | Primer - supP19-1c | — |
| 22 | Primer - SupP19-SacI.r | |
| 23 | CPMV Enhancer Sequence | — |

The present invention will be further illustrated in the following examples. However it is to be understood that these examples are for illustrative purposes only, and should not be used to limit the scope of the present invention in any manner.

### Assembly of Expression Cassettes

Constructs that may be used for the production of VLPs are described in WO 2009/009876, WO 2009/076778 and W02010/003225, and U.S. Provisional Application No. 61/220,161 (filed June 24, 2009), all of which are incorporated herein by reference. Non-limiting examples of constructs may also include those listed in Table 2. Assembly of these constructs is described in WO 2009/009876, WO 2009/076778, WO2010/003225 and U.S. 61/220,161. However other constructs comprising known HAs, including but not limited to, those provided in Table 2, and combined with similar or different regulatory elements and promoters, may also be used for the production of VLPs as described herein.

TABLE 2
Examples of constructs used for hemagglutinin production.

| Cassette | | HA |
|---|---|---|
| number | Corresponding HA | abbreviation |
| 540 | SpPDI-H1 from strain A/New Caledonia/20/99 | H1/NC |
| | (H1N1) | |
| 560 | SpPDI-H1 A/California/4/2009 in | H1/Cal WT |
| | 2X35S/CPMV-HT expression cassette | |
| 580 | SpPDI-H1 A/New Caledonia/20/99 in | H1/NC |
| | 2x35S/CPMV-HT expression cassette | |
| 660 | H5 from strain A/Indonesia/5/2005 (H5N1) | H1/Indo |
| 663 | H5 A/Indonesia/5/2005 | H1/Indo |
| 685 | H5 A/Indonesia/5/2005 in CPMV-HT | H1/Indo |
| | expression cassette | |
| 686 | SpPDI-H5 A/Indonesia/5/2005 in CPMV-HT | H1/Indo |
| | expression cassette | |
| 690 | H1 A/Brisbane/59/07 receptor-binding (RB) | H1/Bris |
| | domain in H5 A/Indonesia/5/05 backbone | |
| 691 | H1 A/Brisbane/59/07 esterase and receptor- | H1/Bris |
| | binding domains (E1-RB-E2) in H5 | |
| | A/Indonesia/5/05 backbone | |
| 696 | H5 A/Indonesia/5/05 receptor-binding (RB) | H1/Indo |
| | domain in H1 A/New Caledonia/20/99 backbone | |
| 732 | H1 A/Brisbane/59/2007 in CPMV-HT | H1/Bris |
| | expression cassette | |
| 733 | SpPDI-H1 A/Brisbane/59/2007 in CPMV-HT | H1/Bris |
| | expression cassette | |
| 734 | H1 A/Brisbane/59/07 receptor-binding (RB) | H1/Bris |
| | domain in H5 A/Indonesia/5/05 backbone in | |
| | CPMV-HT expression cassette | |
| 735 | H3 A/Brisbane/10/2007 in CPMV-HT | H3/Bris |
| | expression cassette | |
| 736 | SpPDI-H3 A/Brisbane/10/2007 in CPMV-HT | H3/Bris |
| | expression cassette | |
| 737 | Assembly of chimeric SpPDI-H3 | H3/Bris-H5/ |
| | A/Brisbane/10/2007 (ectodomain) + H5 | Indo |
| | A/Indonesia/5/2005 (TmD + Cyto tail) in | chimera |
| | CPMV-HT expression cassette | |
| 738 | HA B/Florida/4/2006 in CPMV-HT expression | B/Flo |
| | cassette | |
| 739 | SpPDI-HA B/Florida/4/2006 in CPMV-HT | B/Flo |
| | expression cassette | |
| 745 | SpPDI-HA B/Florida/4/2006 (ectodomain) + H5 | B/Flo |
| | A/Indonesia/5/2005 (TmD + Cyto tail) in | |
| | CPMV-HT expression cassette | |
| 747 | SpPDI-HA B/Florida/4/2006 + H5 | B/Flo |
| | A/Indonesia/5/2005 (TmD + Cyto tail) in | |
| | 2X35S-CPMV-HT expression cassette | |
| 774 | HA of A/Brisbane/59/2007 (H1N1) | H1/Bris |
| 775 | HA of A/Solomon Islands 3/2006 (H1N1) | H1/Solomon |
| 776 | HA of A/Brisbane 10/2007 (H3N2) | H3/Bris |
| 777 | HA of A/Wisconsin/67/2005 (H3N2) | H3/Wisc |
| 778 | HA of B/Malaysia/2506/2004 | B/Malaysia |
| 779 | HA of B/Florida/4/2006 | B/Flo |
| 780 | HA of A/Singapore/1/57 (H2N2) | H2/Sing |
| 781 | HA of A/Anhui/1/2005 (H5N1) | H5/Anhui |
| 782 | HA of A/Vietnam/1194/2004 (H5N1) | H5/Vietnam |
| 783 | HA of A/Teal/HongKong/W312/97 (H6N1) | H6/ |
| | | HongKong |
| 784 | HA of A/Equine/Prague/56 (H7N7) | H7/Prague |
| 785 | HA of A/HongKong/1073/99 (H9N2) | H9/ |
| | | HongKong |
| 787 | H1 A/Brisbane/59/2007 | H1/Bris |
| 790 | H3 A/Brisbane/10/2007 | H3/Bris |
| 798 | HA B/Florida/4/2006 | B/Flo |

### EXAMPLES

### Materials and Methods—Molecular Cloning

CaMV 2X35S-CPMV HT-based expression cassettes

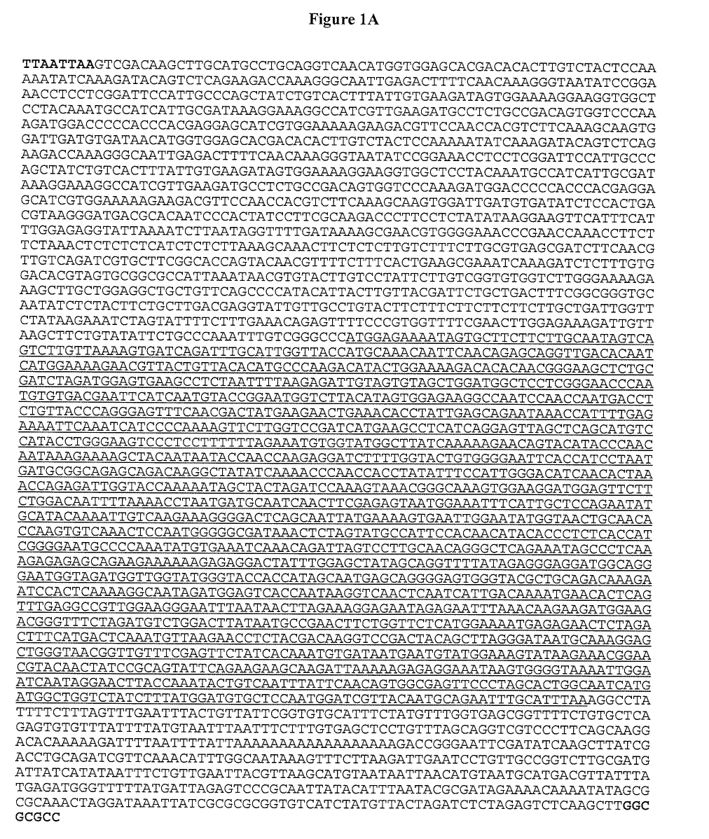

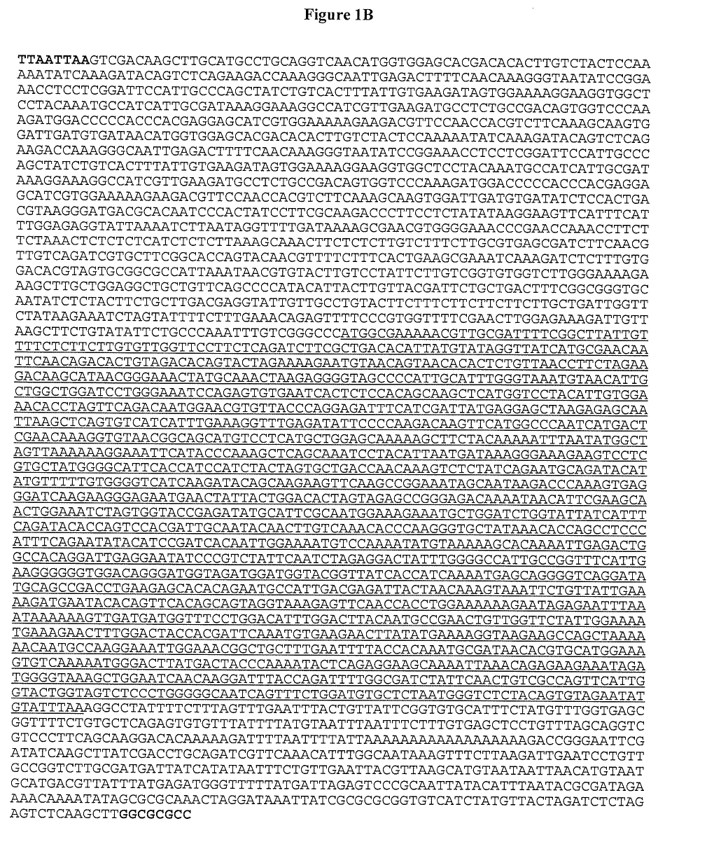

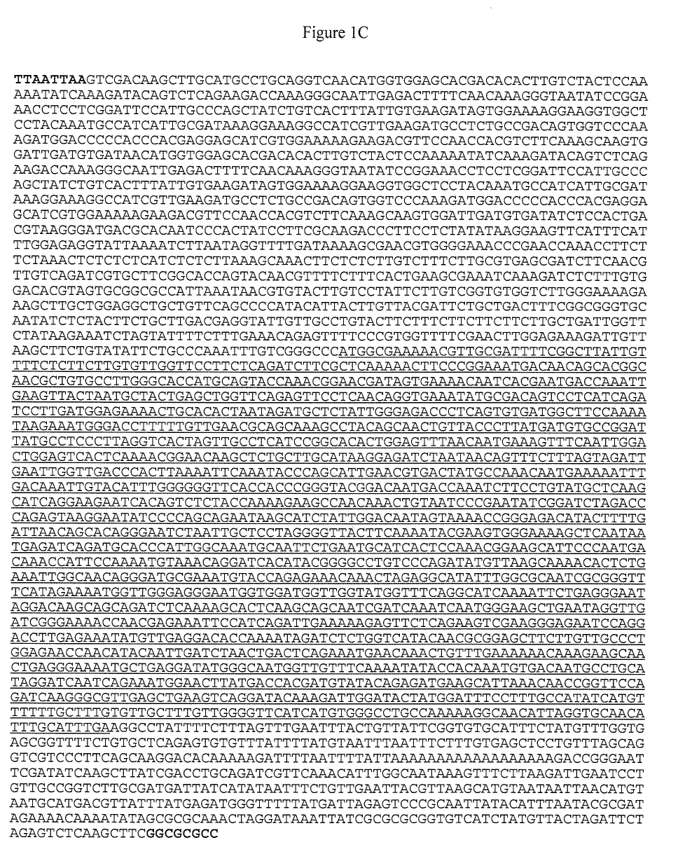

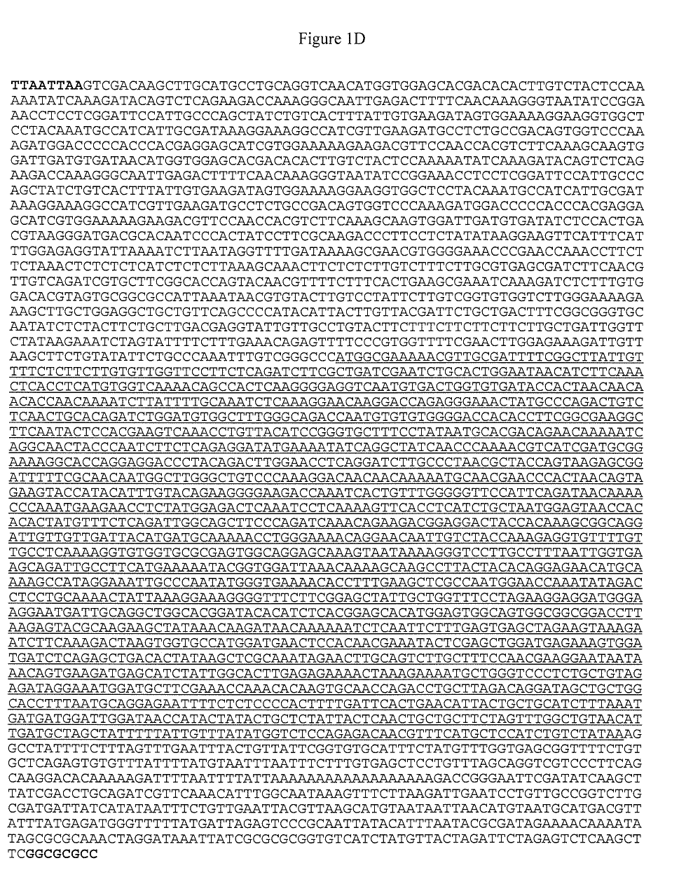

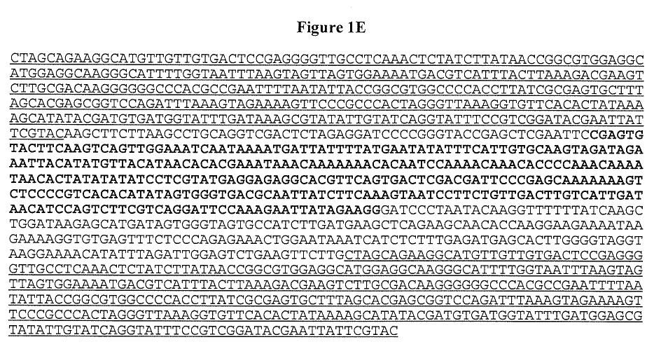

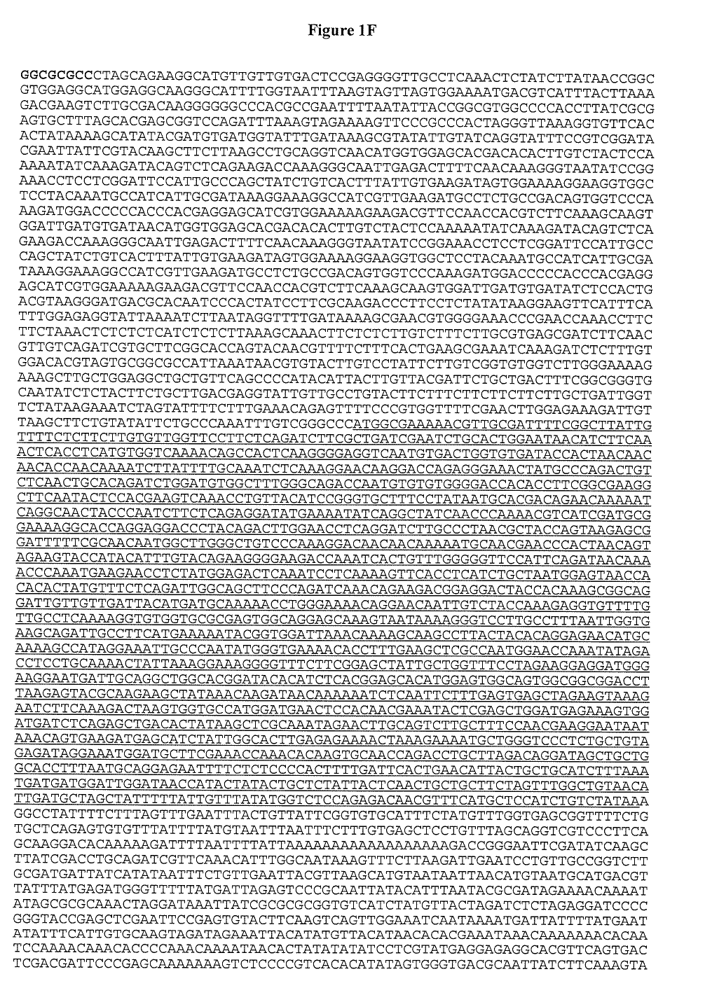

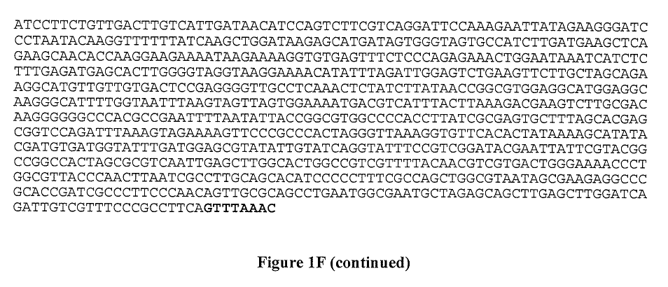

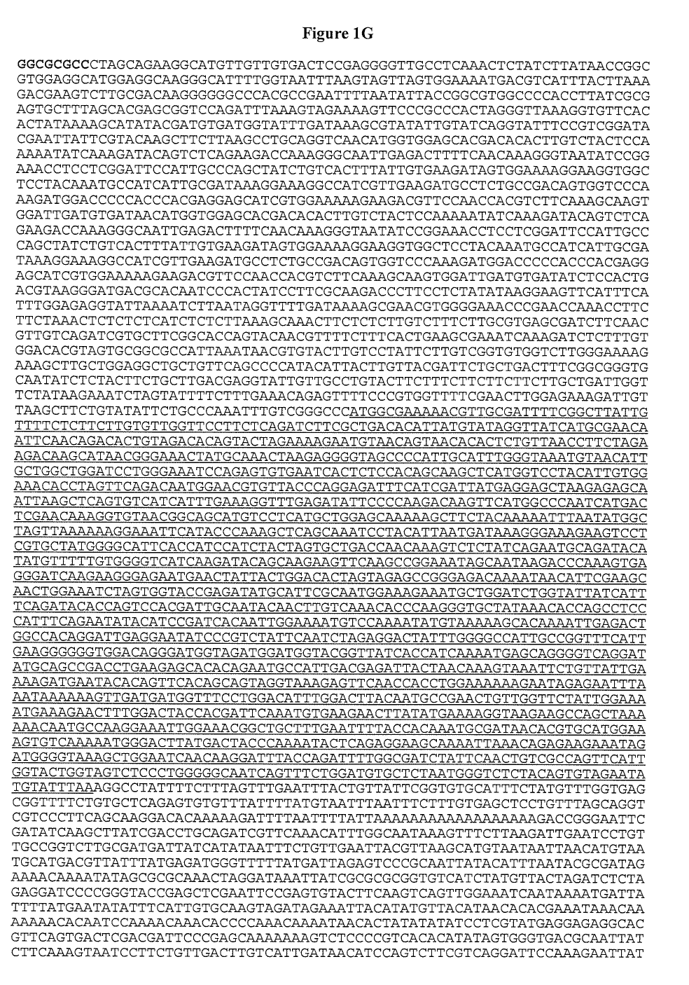

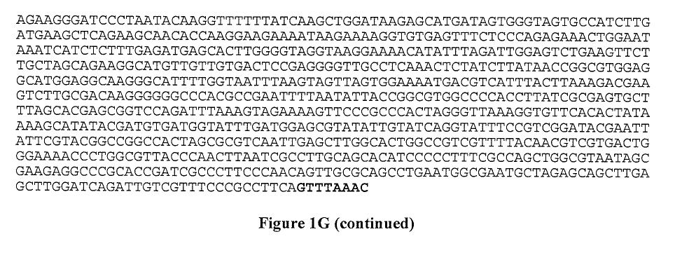

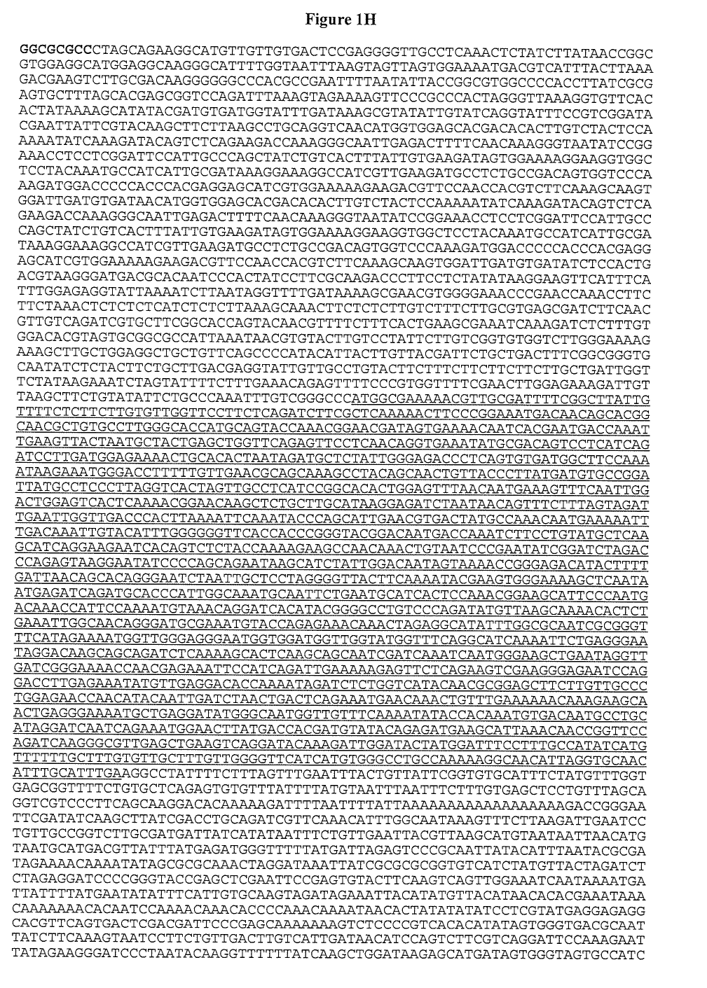

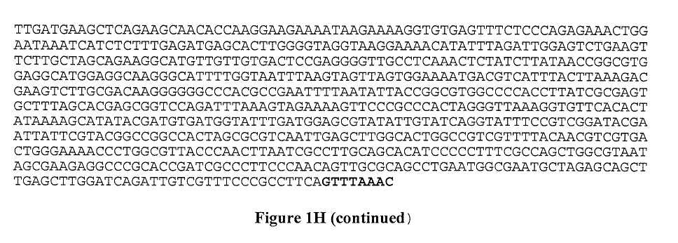

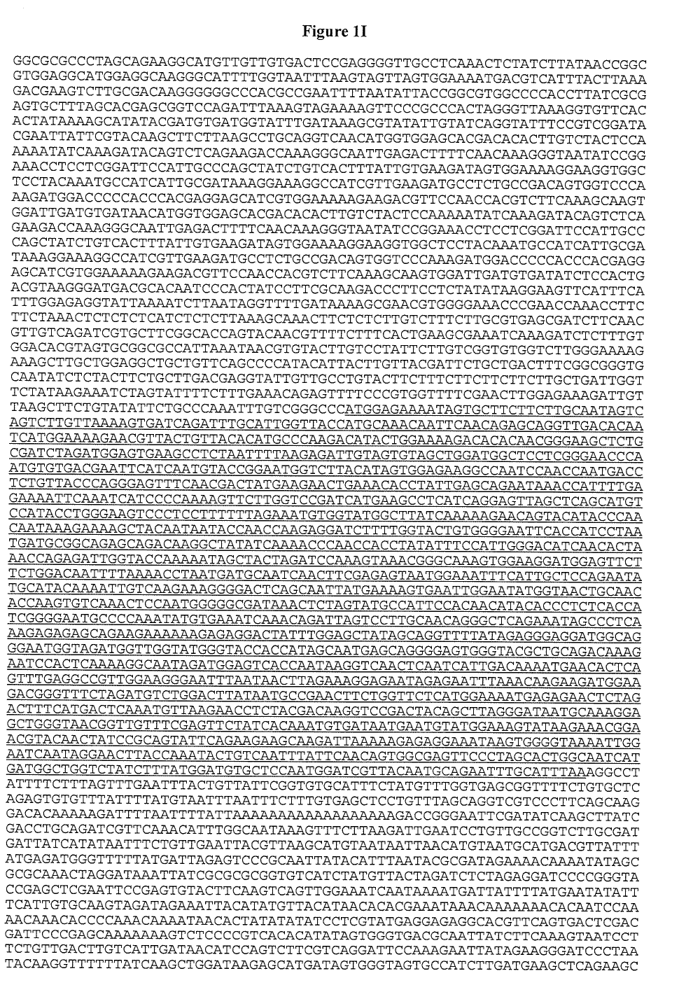

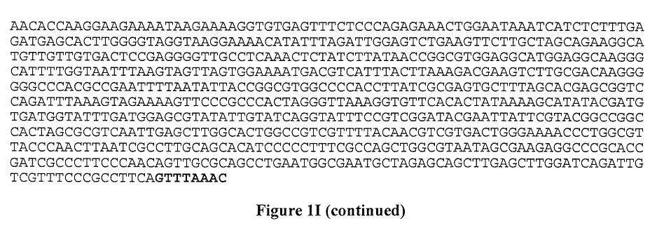

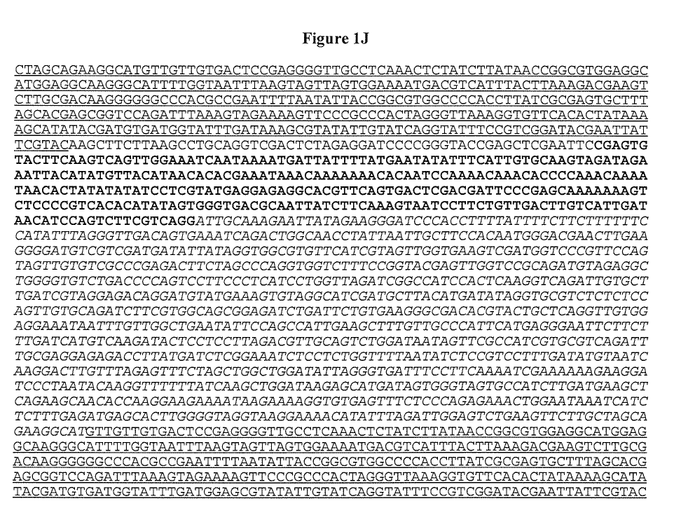

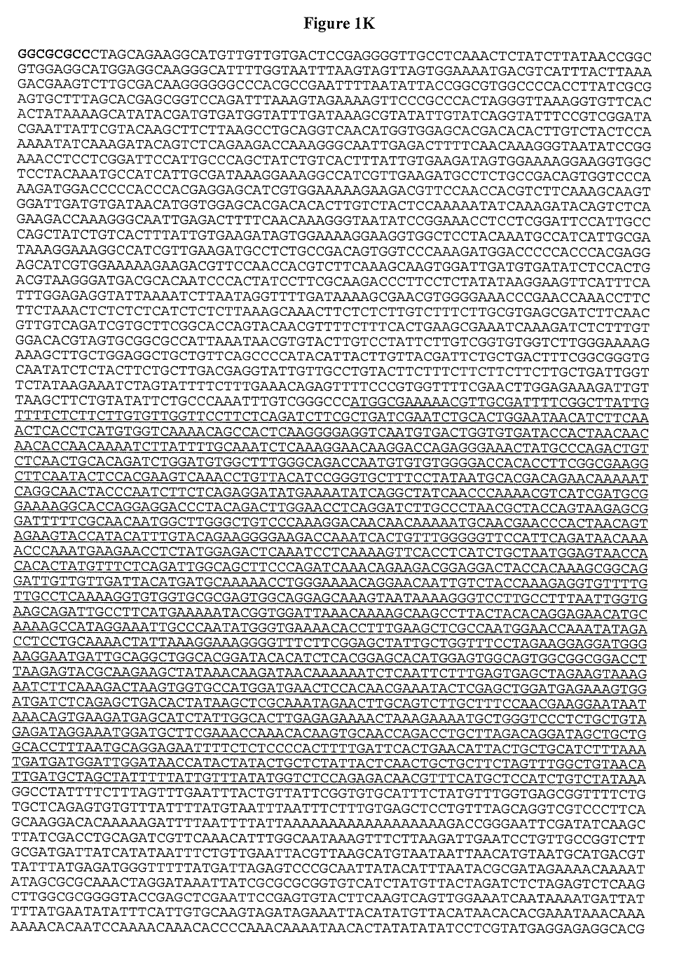

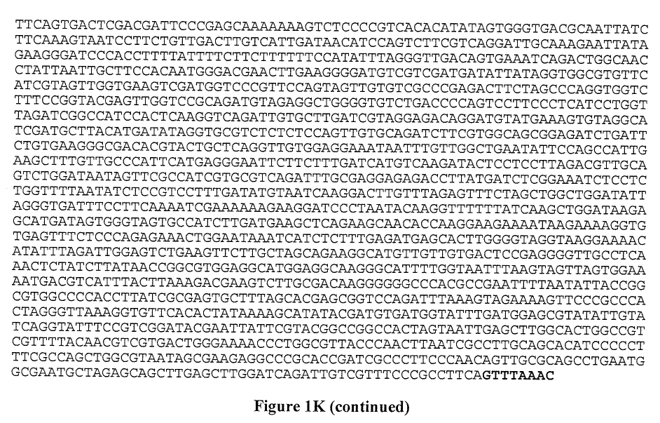

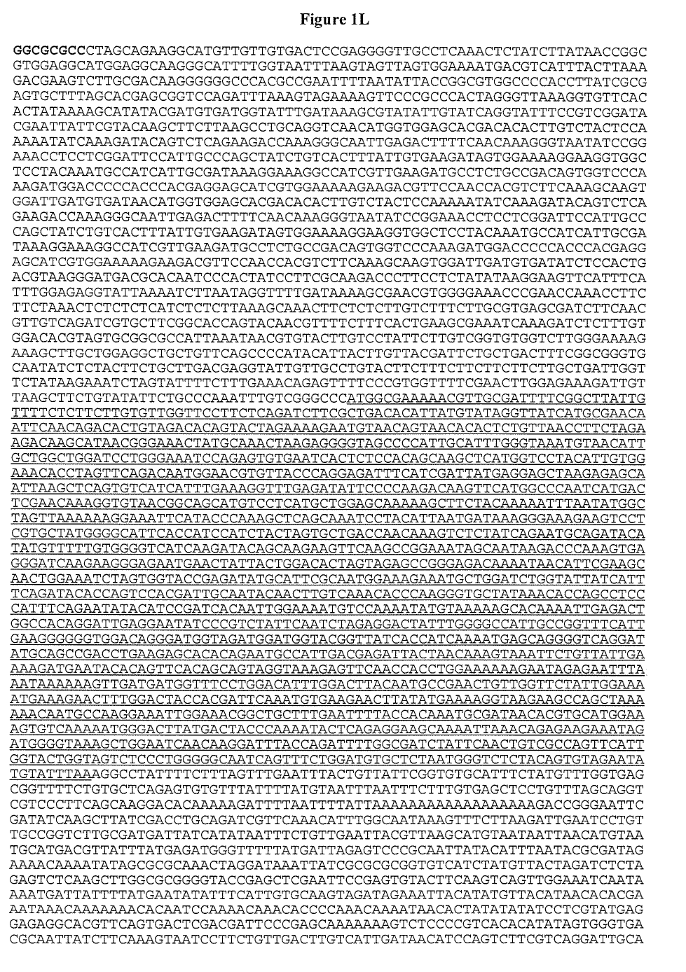

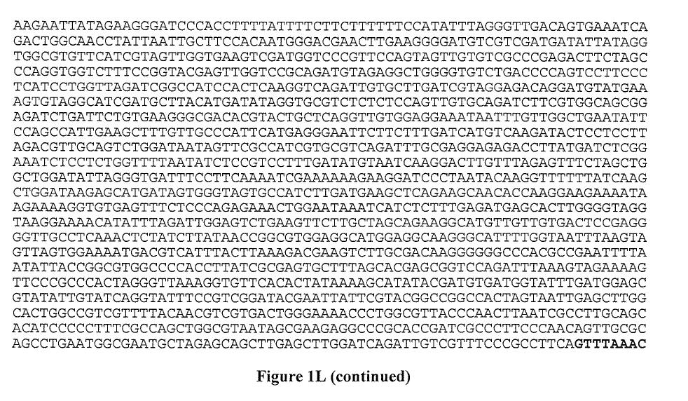

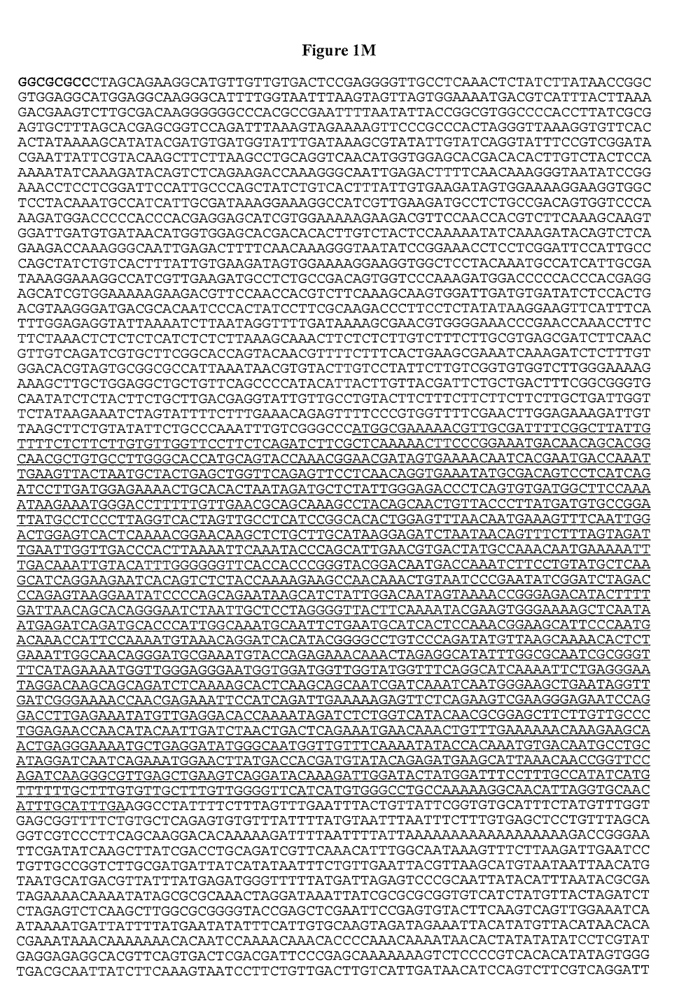

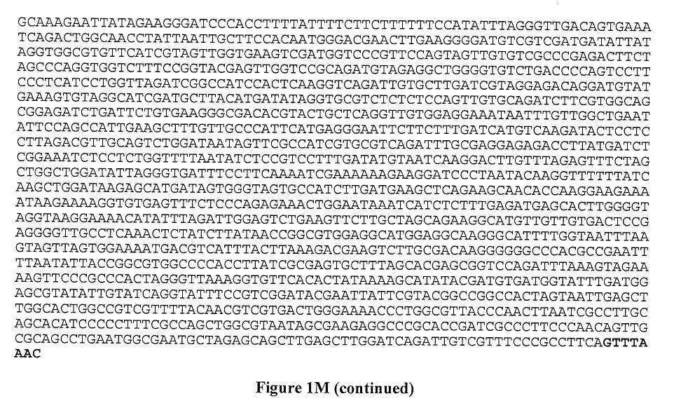

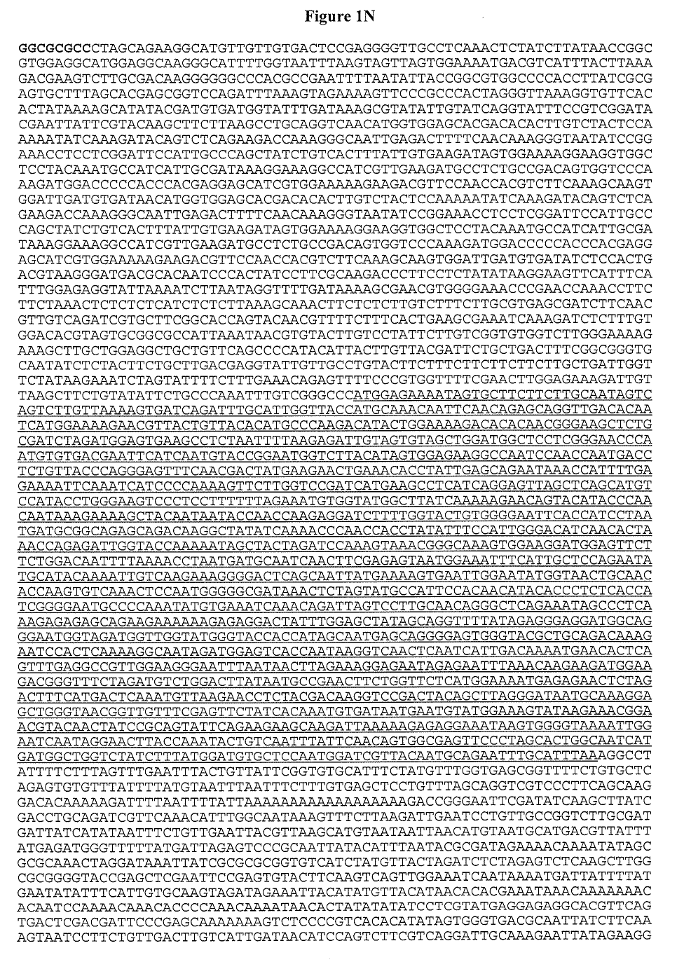

2X35S-CPMV H -Native Sp-HA0 H5 A/Indonesia/5/2005 (construct number 972; SEQ ID NO:1; FIG. 1A). The assembly of construct number 972, comprising the coding region for HA0 from H5 A/Indonesia/5/2005 with its native signal peptide under the control of CaMV 2X35S-CPMV HT expression cassette was described previously (W02010/003225, incorporated herein by reference). The sequence of the resulting expression cassette is presented in FIG. 1A (SEQ ID NO:1).

2X35S-CPMV HT -SpPDI-HA0 H1 A/California/4/2009 (construct number 560; SEQ ID NO:1 2; FIG. 1B). The assembly of construct number 560, comprising the nucleotide sequence encoding alfalfa PDI signal peptide (SpPDI) fused to HA0 from H1 A/California/4/2009 under the control of CaMV 2X355-CPMV-HT was described previously (WO2010/003225). The sequence of the resulting expression cassette is presented in FIG. 1B (SEQ ID NO:2).

2X35S-CPMV HT -SpPDI-HA0 H3 A/Brisbane/10/2007 (construct number 971; SEQ ID NO:3; FIG. 1C). A sequence encoding alfalfa PDI signal peptide fused to HA0 from H3 A/Brisbane/10/2007 was assembled into CaMV 2X35S-CPMV-HT regulatory elements as follows. Construct number 736 was described previously (see PCT Publication No. WO 2010/003225 for assembly and sequence)—briefly, construct 736 was digested with restriction enzymes ApaI (immediately upstream of the ATG) and StuI (immediately downstream of the stop codon) to remove a fragment encoding SpPDI fused to HA0 from H3 A/Brisbane/10/2007. The resulting fragment was cloned into construct 972 (SEQ ID NO:1′; FIG. 1A) previously digested with ApaI and StuI. The resulting construct was given number 971 (SEQ ID NO:3, FIG. 1C).

2X35S-CPMV HT -SpPDI-HA0 B/Florida/4/2006 (construct number 973; SEQ ID NO:4; FIG. 1D). A sequence encoding alfalfa PDI signal peptide fused to HA0 from B/Florida/4/2006 was assembled into to CaMV 2X35S-CPMV-HT as follows. Construct number 739 was described previously (see PCT Publication No. WO 2010/003225 for assembly and sequence)—briefly, construct 739 was digested with restriction enzymes ApaI (immediately upstream of the ATG) and StuI (immediately downstream of the stop codon) to remove a fragment encoding SpPDI fused to HA0 from B/Florida/4/2006. The resulting fragment was cloned into construct 972 previously digested with ApaI and StuI. The resulting construct was given number 973 (SEQ ID NO:4, FIG. 1D).

CaMV 2X35S-CPMV HT-Based Cassettes with BeYDV Amplification Elements

Acceptor plasmid containing LIR-Multiple cloning site (MCS)-SIR+LIR in pCAMBIA based-vector (construct 849; SEQ ID NO:9; FIG. 1E). The long intergenic regions (LIR) and the short intergenic region (SIR) from Bean Yellow dwarf virus (BeYDV) were inserted into pCAMBIA binary vector as follow. A first PCR fragment containing BeYDV LIR sequence was amplified using MfeI-MluI-AscI-LIR.c (SEQ ID NO:5):

CAATTGACGCGTGGCGCGCCCTAGCAGAAGGCATGTTGTTGTGACTCCGAGG

### and SbfI-AflII-HindIII-LIR.r (SEQ ID NO:6):

CCTGCAGGCTTAAGAAGCTTGTACGAATAATTCGTATCCGACGGAA

as primers. The 2963 pb BglII restriction fragment of construct pBYGFP (kindly provided by Dr Hugh Mason, Arizona State University) was gel-extracted and used as template for the first PCR reaction. Vector pBYGFP has been previously described in Huang et al. (Biotechnology and Bioengineering 103: 706-714 (2009)). The resulting fragment was digested with SbfI and cloned into pCAMBIA2300 (Cambia, Canberra, Australia) previously digested with HindIII, treated with T4 DNA polymerase to create blunt end and finally digested with SbfI. The resulting plasmid was named pCAMBIA-LIR. A second fragment containing BeYDV LIR and SIR sequences was amplified using SacI-EcoRI-SIR.c (SEQ ID NO:7)

TACCGAGCTCGAATTCCGAGTGTACTTCAAGTCAGTTGGAAATC

### and SpeI-FseI-LIR.r (SEQ ID NO:8)

ACTAGTGGCCGGCCGTACGAATAATTCGTATCCGACGGAAATACCTGA

as primers and the 2116 pb BglII restriction pBYGFP fragment as template. The resulting fragment was digested with Sad and cloned into pCAMBIA-LIR previously digested with EcoRI, treated with T4 DNA polymerase to create blunt end and finally digested with SacI. The resulting plasmid was named pCAMBIA-LIR-MCS-SIR+LIR. In order to change the orientation of the LIR-MCS-SIR+LIR sequence relative to the orientation of the T-DNA borders, pCAMBIA-LIR-MCS-SIR+LIR was digested with MluI and SpeI restriction enzymes and the resulting fragments were blunt-ended using T4 DNA polymerase and re-ligated. The clones obtained from the transformation of this ligation product were screened for orientation and a clone in which the LIR-MCS-SIR+LIR elements were in Left to Right T-DNA orientation was selected and kept as acceptor construct 849. An annotated LIR-MCS-SIR+LIR sequence is presented in SEQ ID NO:9 (FIG. 1E).

2X35S-CPMV HT -SpPDI-HA0 B/Florida/4/2006 in BeYDV amplification elements (construct number 853; SEQ ID NO:10; FIG. 1F). A sequence encoding alfalfa PDI signal peptide fused to HA0 from B/Florida/4/2006 under the control of CaMV 2X35S-CPMV-HT into the BeYDV amplification elements was assembled as follows. Construct number 973 (SEQ ID NO: 4, FIG. 1D) was digested with SbfI (upstream of the CaMV 2X355 promoter) and XbaI (downstream of the NOS terminator) to remove a fragment containing the whole 2X35S-CPMV HT -SpPDI-HA0 B/Florida/4/2006 expression cassette. The resulting fragment was cloned into construct number 849 (SEQ ID NO:9, FIG. 1E) previously digested with SbfI and XbaI. The resulting construct was given number 853 (SEQ ID NO: 10, FIG. 1F).

2X35S-CPMV HT -SpPDI-HA0 H1 A/California/4/2009 in BeYDV amplification elements (construct number 561; SEQ ID NO:11, FIG. 1G). A sequence encoding alfalfa PDI signal peptide fused to HA0 from H1 A/California/4/2009 under the control of CaMV 2X35S-CPMV-HT into the BeYDV amplification elements was assembled as follows. Construct number 560 (SEQ ID NO:2, FIG. 1B) was digested with SbfI (upstream of the CaMV 2X355 promoter) and StuI (downstream of the Stop codon) to remove a fragment containing the 2X35S-CPMV HT -SpPDI-H1 A/California/4/2009 expression cassette without NOS terminator. The resulting fragment was cloned into construct number 853 (SEQ ID NO: 10, FIG. 1F) previously digested with SbfI and StuI (containing the NOS terminator). The resulting construct was given number 561 (SEQ ID NO:11, FIG. 1G).

2X35S-CPMV HT -SpPDI-HA0 H3 A/Brisbane/10/2007 in BeYDV amplification elements (construct number 851; SEQ ID NO:12, FIG. 1H). A sequence encoding alfalfa PDI signal peptide fused to HA0 from H3 A/Brisbane/10/2007 under the control of CaMV 2X35S-CPMV-HT into the BeYDV amplification elements was assembled as follows. Construct number 971 (SEQ ID NO:3, FIG. 1C) was digested with SbfI (upstream of the CaMV 2X355 promoter) and StuI (downstream of the Stop codon) to remove a fragment containing the 2X35S-CPMV HT -SpPDI-H3 A/Brisbane/10/2007 expression cassette without NOS terminator. The resulting fragment was cloned into construct number 853 (SEQ ID NO: 10, FIG. 1F) previously digested with SbfI and StuI (containing the NOS terminator). The resulting construct was given number 851 (SEQ ID NO: 12, FIG. 1H).

2X35S-CPMV HT -Native Sp-HA0 H5 A/Indonesia/5/2005 in BeYDV amplification elements (construct number 852; SEQ ID NO:13, FIG. 1I). A sequence encoding HA0 from H5 A/Indonesia/5/2005 with is native signal under the control of CaMV 2X35S-CPMV-HT into the BeYDV amplification elements was assembled as follows. Construct number 972 (SEQ ID NO:1 FIG. 1A) was digested with SbfI (upstream of CaMV 2X35S promoter) and StuI (downstream of the Stop codon) to remove a fragment containing the 2X35S-CPMV HT -Native Sp-HA0 H5 A/Indonesia/5/2005 expression cassette without NOS terminator. The resulting fragment was cloned into construct number 853 (SEQ ID NO: 10, FIG. 1F) previously digested with SbfI and StuI (containing the NOS terminator). The resulting construct was given number 852 (SEQ ID NO: 13, FIG. 1I).

Replicase Expression Construct

Replicase expression construct (construct number 834). The plasmid bearing the construct for the expression of BeYDV replicase was kindly provided by Dr Hugh Mason (Arizona State University). Construct number 834 corresponds to plasmid pREP110 described in Huang et al. (2009, Biotechnol. Bioeng. 103, 706-714).

CaMV 2X35S-CPMV HT-based cassettes with BeYDV amplification elements and replicase gene under the control of LIR

Acceptor plasmid containing LIR-Multiple cloning site (MCS)-SIR-replicase-LIR into pCAMBIA based-vector (construct 848; SEQ ID NO:14, FIG. 1J). The BeYDV replicase (C1/C2 with deleted intron) was inserted between BeYDV SIR and LIR repeat as follows. A PCR fragment was amplified using SacI-EcoRI-SIR.c (SEQ ID NO:7) and SpeI-FseI-LIR.r (SEQ ID NO:8) as primers. The 2180 pb SacI-FseI restriction fragment of construct pBYGFP.R (kindly provided by Dr Hugh Mason, Arizona State University) was gel-extracted and used as template for the PCR reaction. Vector pBYGFP.R has been previously described in Huang et al. (Biotechnology and Bioengineering 103: 706-714 (2009)). The resulting fragment was digested with Sad and cloned into construct 849 (SEQ ID NO:9, FIG. 1E) previously digested with MfeI, treated with T4 DNA polymerase to create blunt end and finally digested with SacI. The resulting acceptor construct was given number 848 (SEQ ID NO:14; FIG. 1J).

2X35S-CPMV HT -SpPDI-HA0 B/Florida/4/2006 in BeYDV+replicase amplification system (construct number 475; SEQ ID NO:15, FIG. 1K). A sequence encoding alfalfa PDI signal peptide fused to HA0 from B/Florida/4/2006 under the control of CaMV 2X35S-CPMV-HT into the BeYDV with replicase amplification system was assembled as follows. Construct number 973 (SEQ ID NO:4, FIG. 1D) was digested with SbfI (upstream of the CaMV 2X35S promoter) and XbaI (downstream of the NOS terminator) to remove a fragment containing the whole 2X35S-CPMV HT -SpPDI-HA0 B/Florida/4/2006 expression cassette. The resulting fragment was cloned into construct number 848 (SEQ ID NO:14, FIG. 1J) previously digested with SbfI and XbaI. The resulting construct was given number 475 (SEQ ID NO:15, FIG. 1K).

2X355-CPMV HT -SpPDI-HA0 H1 A/California/4/2009in BeYDV+replicase amplification system (construct number 471; SEQ ID NO:16, FIG. 1L). A sequence encoding alfalfa PDI signal peptide fused to HA0 from HI A/California/4/2009 under the control of CaMV 2X35S-CPMV-HT into the BeYDV with replicase amplification system was assembled as follows. Construct number 560 (SEQ ID NO:2, FIG. 1B) was digested with SbfI (upstream of the CaMV 2X35S promoter) and StuI (downstream of the Stop codon) to remove a fragment containing the 2X35S-CPMV HT -SpPDI-HI A/California/4/2009 expression cassette without NOS terminator. The resulting fragment was cloned into construct number 475 (SEQ ID NO:15, FIG. 1K) previously digested with SbfI and StuI (containing the NOS terminator). The resulting construct was given number 471 (SEQ ID NO:16, FIG. 1L).

2X35S-CPMV HT -SpPDI-HA0 H3 A/Brisbane/10/2007 in BeYDV+replicase amplification system(construct number 473; SEQ ID NO:17, FIG. 1M). A sequence encoding alfalfa PDI signal peptide fused to HA0 from H3 A/Brisbane/10/2007 under the control of CaMV 2X35S-CPMV-HT into the BeYDV with replicase amplification system was assembled as follows. Construct number 971 (SEQ ID NO:3, FIG. 1C) was digested with SbfI (upstream of the CaMV 2X35S promoter) and StuI (downstream of the Stop codon) to remove a fragment containing the 2X35S-CPMV HT -SpPDI-H3 A/Brisbane/10/2007 expression cassette without NOS terminator. The resulting fragment was cloned into construct number 853 (SEQ ID NO:10, FIG. 1F) previously digested with SbfI and StuI (containing the NOS terminator). The resulting construct was given number 473 (SEQ ID NO:17, FIG. 1M).

2X35S-CPMV HT -Native Sp-HA0 H5 A/Indonesia/5/2005 in BeYDV+replicase amplification system(construct number 474; SEQ ID NO:18, FIG. 1N). A sequence encoding HA0 from H5 A/Indonesia/5/2005 with is native signal under the control of CaMV 2X35S-CPMV-HT into the BeYDV with replicase amplification system was assembled as follows. Construct number 972 (SEQ ID NO:1, FIG. 1A) was digested with SbfI (upstream of the CaMV 2X35S promoter) and StuI (downstream of the Stop codon) to remove a fragment containing the 2X35S-CPMV HT -Native Sp-HA0 H5 A/Indonesia/5/2005 expression cassette without NOS terminator. The resulting fragment was cloned into construct number 853 (SEQ ID NO:10, FIG. 1F) previously digested with SbfI and StuI (containing the NOS terminator). The resulting construct was given number 474 (SEQ ID NO:18, FIG. 1N).

Plastocyanin-P19 (construct number R472). The construction of p19 is described in WO 2010/0003225 (which is incorporated herein by reference). Briefly, the coding sequence of p19 protein of tomato bushy stunt virus (TBSV) was linked to the alfalfa plastocyanin expression cassette by the PCR-based ligation method presented in Darveau et al. (Methods in Neuroscience 26: 77-85(1995)). In a first round of PCR, a segment of the plastocyanin promoter was amplified using primers Plasto-443c (SEQ ID NO:19):

GTATTAGTAATTAGAATTTGGTGTC

and supP19-plasto.r (SEQ ID NO:20)

CCTTGTATAGCTCGTTCCATTTTCTCTCAAGATG

with construct 660 (described in WO 2010/0003225, which is incorporated herein by reference) as template. In parallel, another fragment containing the coding sequence of p19 was amplified with primers supP19-1c (SEQ ID NO:21):

ATGGAACGAGCTATACAAGG

### and SupP19-SacI.r (SEQ ID NO:22):

AGTCGAGCTCTTACTCGCTTTCTTTTTCGAAG

using construct 35S:p19 as described in Voinnet et al. (2003, The Plant Journal 33: 949-956) as template. Amplification products were then mixed and used as template for a second round of amplification (assembling reaction) with primers Plasto-443c and SupP19-SacI.r. The resulting fragment was digested with BamHI (in the plastocyanin promoter) and SacI (at the end of the p19 coding sequence) and cloned into construct number 660, previously digested with the same restriction enzymes to give construct number R472. The plasmids were used to transform Agrobacteium tumefaciens (AGL1; ATCC, Manassas, Va. 20108, USA) by electroporation (Mattanovich et al., 1989, Nucleic Acids Res. 17, 6747). The integrity of all A. tumefaciens strains were confirmed by restriction mapping. The A. tumefaciens strain comprising R472 is termed “AGL1/R472”.

### Preparation of Plant Biomass, Inoculum, Agroinfiltration, and Harvesting

Nicotiana benthamiana plants were grown from seeds in flats filled with a commercial peat moss substrate. The plants were allowed to grow in the greenhouse under a 16/8 photoperiod and a temperature regime of 25° C. day/20° C. night. Three weeks after seeding, individual plantlets were picked out, transplanted in pots and left to grow in the greenhouse for three additional weeks under the same environmental conditions.

Agrobacteria transfected with each construct were grown in a YEB medium supplemented with 10 mM 2-(N-morpholino)ethanesulfonic acid (MES), 20 μM acetosyringone, 50 μg/mlkanamycin and 25 μg/ml of carbenicillin pH5.6 until they reached an OD₆₀₀ between 0.6 and 1.6. Agrobacterium suspensions were centrifuged before use and resuspended in infiltration medium (10 mM MgCl₂ and 10 mM MES pH 5.6). and stored overnight at 4° C. On the day of infiltration, culture batches were diluted in 2.5 culture volumes and allowed to warm before use. Whole plants of N. benthamiana were placed upside down in the bacterial suspension in an air-tight stainless steel tank under a vacuum of 20-40 Torr for 2-min. Plants were returned to the greenhouse for a 2-6 day incubation period until harvest. Unless otherwise specified, all infiltrations were performed as co-infiltration with strain AGL1/R472 in a 1:1 or 1:1:1 ratio when co-infiltrated with AGL1/834 for the co-expression of the BeYDV replicase.

A. tumefaciens strains comprising the various constructs as described herein are referred to by using an “AGL1”prefix. For example A. tumefaciens comprising construct number 972 (FIG. 1A), is termed “AGL1/972”.

### Leaf Sampling and Total Protein Extraction

Following incubation, the aerial part of plants was harvested, frozen at −80° C., crushed into pieces. Total soluble proteins were extracted by homogenizing (Polytron) each sample of frozen-crushed plant material in 3 volumes of cold 50 mM Tris pH 8.0, 0.15 M NaCl, 0.1% Triton X-100 and 1 mM phenylmethanesulfonyl fluoride. After homogenization, the slurries were centrifuged at 10,000 g for 10 min at 4° C. and these clarified crude extracts (supernatant) kept for analyses.

### Protein Analysis and Immunoblotting

The total protein content of clarified crude extracts was determined by the Bradford assay (Bio-Rad, Hercules, Calif.) using bovine serum albumin as the reference standard. Proteins were separated by SDS-PAGE and electrotransferred onto polyvinylene difluoride (PVDF) membranes (Roche Diagnostics Corporation, Indianapolis, Ind.) for immunodetection. Prior to immunoblotting, the membranes were blocked with 5% skim milk and 0.1% Tween-20 in Tris-buffered saline (TBS-T) for 16-18h at 4° C.

Immunoblotting was performed by incubation with a suitable primary antibody (Table 3), in 2 μg/ml in 2% skim milk in TBS-Tween 20 0.1%. Secondary antibodies used for chemiluminescence detection were as indicated in Table 3, diluted as indicated in 2% skim milk in TBS-Tween 20 0.1%. Immunoreactive complexes were detected by chemiluminescence using luminol as the substrate (Roche Diagnostics Corporation). Horseradish peroxidase—enzyme conjugation of human IgG antibody was carried out by using the EZ-Link Plus® Activated Peroxidase conjugation kit (Pierce, Rockford, Ill.).

TABLE 3
Electrophoresis conditions, antibodies, and dilutions
for immunoblotting of expressed proteins.

| HA | | Electrophoresis | Primary | | Secondary | |
|---|---|---|---|---|---|---|
| subtype | Influenza strain | condition | antibody | Dilution | antibody | Dilution |
| H1 | A/California/04/09 | Reducing | FII | 4 μg/ml | Goat anti- | 1:10 000 |
| | (H1N1) | | 10-I50F | | mouse (JIR | |
| | | | | | 115-035-146) | |
| H3 | A/Brisbane/10/2007 | Non-Reducing | TGA | 1:4000 | Rabbit anti- | 1:10 000 |
| | (H3N2) | | AS393 | | sheep (JIR | |
| | | | | | 313-035-045) | |
| H5 | A/Indonesia/5/2005 | Reducing | ITC | 1:4000 | Goat anti- | 1:10 000 |
| | (H5N1) | | IT-003- | | rabbit (JIR | |
| | | | 005V | | 111-035-144) | |
| B | B/Florida/4/2006 | Non-Reducing | NIBSC | 1:2000 | Rabbit anti- | 1:10 000 |
| | | | 07/356 | | sheep (JIR | |
| | | | | | 313-035-045) | |

FII: Fitzgerald Industries International, Concord, MA, USA;
NIBSC: National Institute for Biological Standards and Control;
JIR: Jackson ImmunoResearch, West Grove, PA, USA;
ITC: Immune Technology Corporation, Woodside, NY, USA;
TGA: Therapeutic Goods Administration, Australia.

### EXAMPLE 1

### Combining CPMV-HT Expression System with BeYDV Amplification System

Sainsbury and Lomonossoff have developed an efficient system for the expression of recombinant proteins in plants (2008, Plant Physiology 148, 1212-1218). As taught in WO 2009/087391, the system uses untranslated regions (UTRs) of the genomic RNA 2 of the cowpea mosaic virus (CPMV) in which the two first translation initiation codons found in the 5′leader sequence have been deleted. When combined to the CaMV 35S promoter and the nopaline synthase (NOS) terminator, the modified CPMV UTRs were shown to enhance translation of the flanking coding region. The CPMV-based expression system was named CPMV-HT (hyperanslatable).

WO 2010 /025285 teaches that the DNA amplification system based on the replication machinery of the bean yellow dwarf virus (BeYDV) efficiently increases transient expression level of recombinant proteins in plants.

The CPMV-HT expression system for the production of influenza hemagglutinin (HA) by agroinfiltration in Nicotiana benthamiana was found to produce high levels of HA accumulation in several but not all of the HA types that were tested (data not shown). It was also observed that the accumulation level of expressible HA could be enhanced by replacing the CaMV 35S promoter by a modified version, named double CaMV 35S promoter bearing a duplication of upstream regulatory element (data not shown).

To further enhance HA accumulation levels, DNA constructs were prepared that combined the CPMV-HT expression system with the BeYDV amplification system. The assembly of the constructs is described in the materials and methods section (above; see FIG. 1A-1M and Table 1). A schematic representation of the constructs tested is presented in FIG. 2. The resulting constructs were compared to their CPMV-HT-based counterparts for their efficacy to drive high accumulation level of HA in agroinfiltrated plants. Plants producing HA under the control of CPMV-HT were harvested and frozen 5-6 days post infiltration whereas the plants producing HA under the control of CPMV-HT+BeYDV were harvested and frozen 3-4 days post infiltration. On the day of analysis, total soluble proteins were extracted from frozen biomasses and HA accumulation level was analyzed by Western blot as described above.

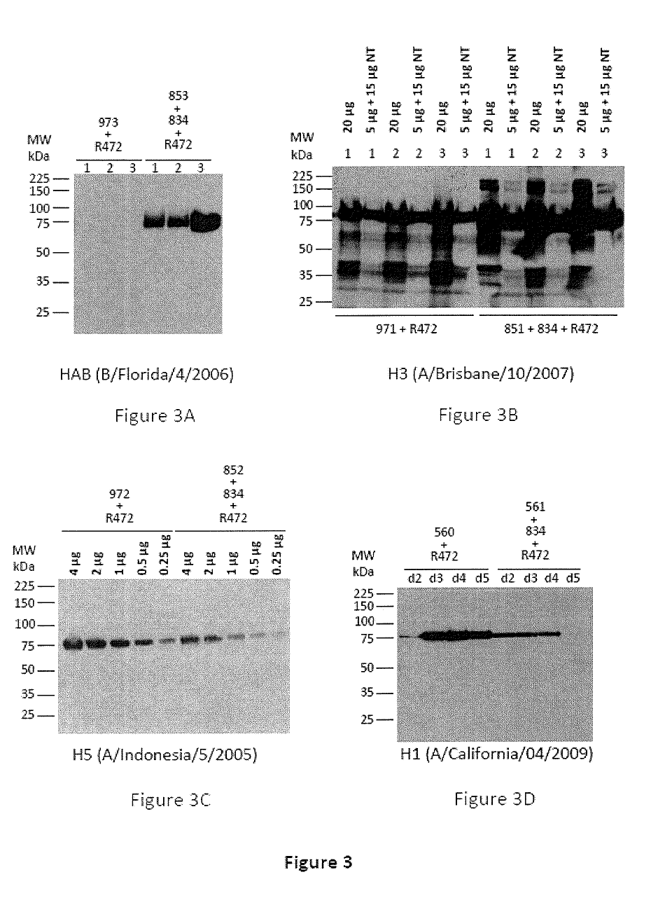

Western blot analysis of HA accumulation level in the biomass showed that a high level of HA from type B virus (from influenza B/Florida/4/2006) was detected in the plants transformed with constructs combining the BeYDV amplification system to the CPMV-HT expression system (AGL1/853; see FIG. 1F+AGL1/834+AGL1/R472), contrasting with the non-detectable expression of the same influenza B virus HA when using the CPMV-HT system alone (AGL1/973; see FIG. 1D+AGL1/R472; FIG. 3A). A comparison of the same expression strategies for H3 (A/Brisbane/10/2007; H3N2) indicates that although CPMV-HT alone drives detectable expression of H3 (AGL1/971; see FIG. 1C+AGL1/R472), the combination of the BeYDV amplification system to CPMV-HT further increased H3 accumulation level (AGL1/851; see FIG. 1H+AGL1/834+AGL1/R472; FIG. 3B).

With H5 from influenza A/Indonesia/05/2005, higher accumulation was observed when using the CPMV-HT expression system alone (AGLI/972; see FIG. 1A+AGL1/R472) than when compared with the constructs combining the BeYDV amplification system to the CPMV-HT expression system (AGLI/852; FIG. 11+AGL1/834+AGL1/R472). This was particularly visible when comparing the 0.25 μg to 1 μg loads (FIG. 3C). H1 accumulation from strain A/California/04/2009 (H1N1), was lower in plants transformed with AGL1/561 (see FIG. 1G)+AGLI/834+AGL1/R472 (H1 under the control of CPMV-HT+BeYDV), when compared with plants transformed with AGL1/560 (; see FIG. 1B)+AGL1/R472 (H1 under the control of CPMV-HT alone (see FIG. 3D). The accumulation profile of H1 was further studied to ensure that the timing of the peak of accumulation did not differ from that of other HAs tested. Maximum accumulation of H1 was observed between days 3 and 5 post infiltration when using the CPMV-HT expression system (AGLI/560+AGL1/R472)whereas the combined CPMV-HT/BeYDV system (AGLI/561+AGL1/834+AGL1/R472) resulted in lower H1 accumulation between days 2 and 4 (FIG. 3D). These results indicate that the combination of BeYDV amplification system to the CPMV-HT-based expression system is not always beneficial and in some combinations, the combination may reduce HA accumulation compared to the use of a CPMV-HT expression system alone.

### EXAMPLE 2

### Producing HA in Plants Using Combined CPMV-HT Expression System and BeYDV Amplification System in a Single Plasmid

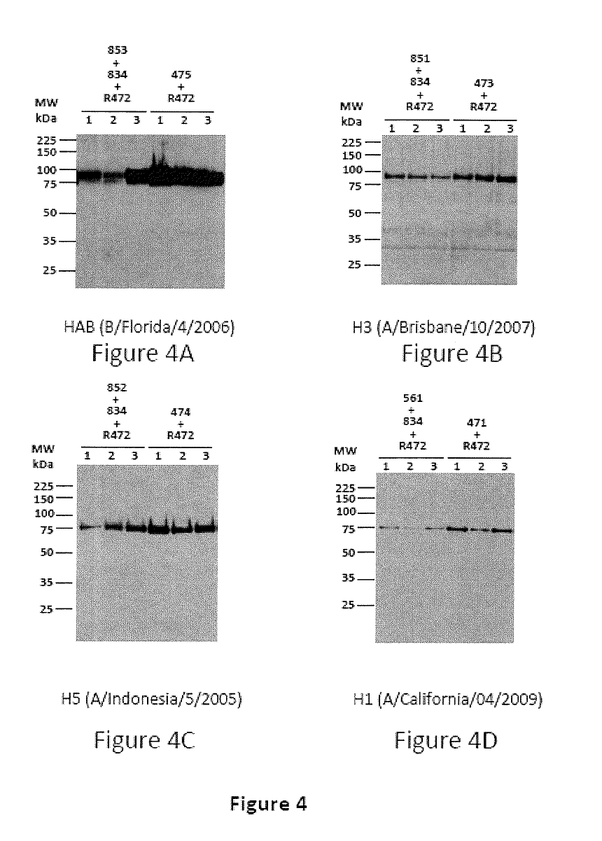

WO 2010/025285 teaches the use of a BeYDV-based DNA amplification system assembled in a single vector. In this system, the replicase genes (C1 and C2) are under the control of the viral LIR promoter. The results presented showed that the single vector BeYDV system was equivalent to the BeYDV system with the replicase genes on a separate plasmid. We have tested if a CPMV-HT+BeYDV expression system would be efficient for HA expression when assembled on a single vector. A schematic representation of the single vector for HA and replicase expression is presented in FIG. 2D. In all cases tested, the single vector system bearing the C1/C2 genes (i.e. replicase in cis) under the control of the viral LIR promoter produced more HA than the dual vector system in which the replicase genes are on a separate plasmid (replicase in trans). Results are presented in FIGS. 4A-4D.

It is contemplated that any embodiment discussed in this specification can be implemented or combined with respect to any method or composition of the invention, and vice versa. Furthermore, compositions of the invention can be used to achieve methods of the invention.

All citations are herein incorporated by reference, as if each individual publication was specifically and individually indicated to be incorporated by reference herein and as though it were fully set forth herein. Citation of references herein is not to be construed nor considered as an admission that such references are prior art to the present invention.

Any terms not directly defined herein shall be understood to have the meanings commonly associated with them as understood within the art of the invention. Certain terms are discussed below, or elsewhere in the specification, to provide additional guidance to the practitioner in describing the devices, methods and the like of embodiments of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples in the specification, including examples of terms, is for illustrative purposes only and does not limit the scope and meaning of the embodiments of the invention herein.

The invention includes all embodiments, modifications and variations substantially as hereinbefore described and with reference to the examples and figures. It will be apparent to persons skilled in the art that a number of variations and modifications can be made without departing from the scope of the invention as defined in the claims. Examples of such modifications include the substitution of known equivalents for any aspect of the invention in order to achieve the same result in substantially the same way.

1-44. (canceled)

45. A plant expression system comprising a first nucleic acid sequence comprising a regulatory region, operatively linked with a one or more than one comovirus enhancer, a nucleotide sequence encoding an influenza hemagglutinin wherein the influenza hemagglutinin is selected from influenza type B hemagglutinin, influenza type A subtype 113 hemagglutinin, and influenza type A subtype H5 hemagglutinin, and one or more than one geminivirus amplification element, and a second nucleic acid sequence encoding a geminivirus replicase.

46. The plant expression system of claim 45, wherein the regulatory region is selected from a plastocyanin promoter, a CaMV 35S promoter, a 2× CaMV35S promoter, a CAS promoter, a RbcS promoter, a Ubi promoter, and an actin promoter.

47. The plant expression system of claim 45, wherein the one or more than one comovirus enhancer is a comovirus 5′ UTR.

48. The plant expression system of claim 47, wherein the comovirus 5′ UTR is a Cowpea Mosaic Virus (CPMV) 5′UTR.

49. The plant expression system of claim 48, wherein the Cowpea Mosaic Virus (CPMV) 5′UTR comprises the nucleotide sequence of SEQ ID NO:23.

50. The plant expression system of claim 47, wherein the one or more than one comovirus enhancer further comprises a comovirus 3′ UTR.

51. The plant expression system of claim 50, wherein the comovirus 3′ UTR is selected from a Cowpea Mosaic Virus 3′ UTR, and a plastocyanin 3′ UTR.

52. The plant expression system of claim 45, wherein the first nucleic acid sequence and the second nucleic acid sequence are located on the same nucleic acid molecule, or they are located on different DNA molecules.

53. The plant expression system of claim 45, wherein the one or more than one geminivirus amplification element is selected from a Bean Yellow Dwarf Virus long intergenic region (BeYDV LIR), and a BeYDV short intergenic region (BeYDV SIR).

54. The plant expression system of claim 45, wherein the influenza hemagglutinin comprises a native signal peptide sequence, or a non-native signal peptide.

55. The plant expression system of claim 45, further comprising a third nucleic acid sequence, the third nucleic acid sequence encoding a suppressor of silencing.

56. The plant expression system of claim 55, wherein the suppressor of silencing is selected from the group HcPro and p19.

57. The plant expression system of claim 55, further comprising a fourth nucleic acid sequence, the fourth nucleic acid sequence encoding a chaperone protein.

58. The plant expression system of claim 57, wherein the chaperone protein is selected from the group Hsp40 and Hsp70.

59. A method of producing an influenza hemagglutinin or an influenza virus like particle (VLP) in a plant or in a portion of a plant, the method comprising introducing into the plant or in the portion of a plant the plant expression system of claim 1, and incubating the plant or the portion of a plant under conditions that permit expression of the nucleotide sequence encoding the influenza hemagglutinin, thereby producing the influenza hemagglutinin or the influenza VLP.

60. The method of claim 59, wherein in the step of introducing, the first nucleic acid sequence and the second nucleic acid sequence are located on the same molecule, or the first nucleic acid sequence and the second nucleic acid sequence are located on different molecules.

61. The method of claim 60, wherein the first nucleic acid sequence and the second nucleic acid sequence are located on different molecules, and the different molecules are introduced in the plant or in the portion of a plant at the same time.

62. The method of claim 59, wherein in the step of incubating, the expression of the nucleotide sequence encoding the influenza hemagglutinin is a transient expression.

63. The method of claim 59, further comprising a step of harvesting the plant or the portion of a plant, thereby obtaining a harvested plant or portion of a plant containing the influenza VLP.

64. The method of claim 63, further comprising a step of isolating the influenza VLP from the harvested plant or portion of a plant containing the influenza VLP, thereby obtaining an isolated influenza VLP.


SEQUENCE LISTING (23 sequences)

SEQ ID NO: 1 - DNA, 3505 bp, Artificial sequence
  Synthesized expression cassette from PacI site
ttaattaagtcgacaagcttgcatgcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccatggagaaaatagtgcttcttcttgcaatagtcagtcttgttaaaagtgatcagatttgcattggttaccatgcaaacaattcaacagagcaggttgacacaatcatggaaaagaacgttactgttacacatgcccaagacatactggaaaagacacacaacgggaagctctgcgatctagatggagtgaagcctctaattttaagagattgtagtgtagctggatggctcctcgggaacccaatgtgtgacgaattcatcaatgtaccggaatggtcttacatagtggagaaggccaatccaaccaatgacctctgttacccagggagtttcaacgactatgaagaactgaaacacctattgagcagaataaaccattttgagaaaattcaaatcatccccaaaagttcttggtccgatcatgaagcctcatcaggagttagctcagcatgtccatacctgggaagtccctccttttttagaaatgtggtatggcttatcaaaaagaacagtacatacccaacaataaagaaaagctacaataataccaaccaagaggatcttttggtactgtggggaattcaccatcctaatgatgcggcagagcagacaaggctatatcaaaacccaaccacctatatttccattgggacatcaacactaaaccagagattggtaccaaaaatagctactagatccaaagtaaacgggcaaagtggaaggatggagttcttctggacaattttaaaacctaatgatgcaatcaacttcgagagtaatggaaatttcattgctccagaatatgcatacaaaattgtcaagaaaggggactcagcaattatgaaaagtgaattggaatatggtaactgcaacaccaagtgtcaaactccaatgggggcgataaactctagtatgccattccacaacatacaccctctcaccatcggggaatgccccaaatatgtgaaatcaaacagattagtccttgcaacagggctcagaaatagccctcaaagagagagcagaagaaaaaagagaggactatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcaatcattgacaaaatgaacactcagtttgaggccgttggaagggaatttaataacttagaaaggagaatagagaatttaaacaagaagatggaagacgggtttctagatgtctggacttataatgccgaacttctggttctcatggaaaatgagagaactctagactttcatgactcaaatgttaagaacctctacgacaaggtccgactacagcttagggataatgcaaaggagctgggtaacggttgtttcgagttctatcacaaatgtgataatgaatgtatggaaagtataagaaacggaacgtacaactatccgcagtattcagaagaagcaagattaaaaagagaggaaataagtggggtaaaattggaatcaataggaacttaccaaatactgtcaatttattcaacagtggcgagttccctagcactggcaatcatgatggctggtctatctttatggatgtgctccaatggatcgttacaatgcagaatttgcatttaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgcc

SEQ ID NO: 2 - DNA, 3520 bp, Artificial Sequence
  Synthesized expression cassette 560 from PacI site
ttaattaagtcgacaagcttgcatgcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccatggcgaaaaacgttgcgattttcggcttattgttttctcttcttgtgttggttccttctcagatcttcgctgacacattatgtataggttatcatgcgaacaattcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacacctagttcagacaatggaacgtgttacccaggagatttcatcgattatgaggagctaagagagcaattaagctcagtgtcatcatttgaaaggtttgagatattccccaagacaagttcatggcccaatcatgactcgaacaaaggtgtaacggcagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccatctactagtgctgaccaacaaagtctctatcagaatgcagatacatatgtttttgtggggtcatcaagatacagcaagaagttcaagccggaaatagcaataagacccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcaaacacccaagggtgctataaacaccagcctcccatttcagaatatacatccgatcacaattggaaaatgtccaaaatatgtaaaaagcacaaaattgagactggccacaggattgaggaatatcccgtctattcaatctagaggactatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaattctgttattgaaaagatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaatgtgaagaacttatatgaaaaggtaagaagccagctaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaactgtcgccagttcattggtactggtagtctccctgggggcaatcagtttctggatgtgctctaatgggtctctacagtgtagaatatgtatttaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgcc

SEQ ID NO: 3 - DNA, 3523 bp, Artificial Sequence
  Synthesized expression cassette 971 from PacI site
ttaattaagtcgacaagcttgcatgcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccatggcgaaaaacgttgcgattttcggcttattgttttctcttcttgtgttggttccttctcagatcttcgctcaaaaacttcccggaaatgacaacagcacggcaacgctgtgccttgggcaccatgcagtaccaaacggaacgatagtgaaaacaatcacgaatgaccaaattgaagttactaatgctactgagctggttcagagttcctcaacaggtgaaatatgcgacagtcctcatcagatccttgatggagaaaactgcacactaatagatgctctattgggagaccctcagtgtgatggcttccaaaataagaaatgggacctttttgttgaacgcagcaaagcctacagcaactgttacccttatgatgtgccggattatgcctcccttaggtcactagttgcctcatccggcacactggagtttaacaatgaaagtttcaattggactggagtcactcaaaacggaacaagctctgcttgcataaggagatctaataacagtttctttagtagattgaattggttgacccacttaaaattcaaatacccagcattgaacgtgactatgccaaacaatgaaaaatttgacaaattgtacatttggggggttcaccacccgggtacggacaatgaccaaatcttcctgtatgctcaagcatcaggaagaatcacagtctctaccaaaagaagccaacaaactgtaatcccgaatatcggatctagacccagagtaaggaatatccccagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaattctgaatgcatcactccaaacggaagcattcccaatgacaaaccattccaaaatgtaaacaggatcacatacggggcctgtcccagatatgttaagcaaaacactctgaaattggcaacagggatgcgaaatgtaccagagaaacaaactagaggcatatttggcgcaatcgcgggtttcatagaaaatggttgggagggaatggtggatggttggtatggtttcaggcatcaaaattctgagggaataggacaagcagcagatctcaaaagcactcaagcagcaatcgatcaaatcaatgggaagctgaataggttgatcgggaaaaccaacgagaaattccatcagattgaaaaagagttctcagaagtcgaagggagaatccaggaccttgagaaatatgttgaggacaccaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagagatgaagcattaaacaaccggttccagatcaagggcgttgagctgaagtcaggatacaaagattggatactatggatttcctttgccatatcatgttttttgctttgtgttgctttgttggggttcatcatgtgggcctgccaaaaaggcaacattaggtgcaacatttgcatttgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagattctagagtctcaagcttcggcgcgcc

SEQ ID NO: 4 - DNA, 3580 bp, Artificial Sequence
  Synthesized expression cassette 973 from PacI site
ttaattaagtcgacaagcttgcatgcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccatggcgaaaaacgttgcgattttcggcttattgttttctcttcttgtgttggttccttctcagatcttcgctgatcgaatctgcactggaataacatcttcaaactcacctcatgtggtcaaaacagccactcaaggggaggtcaatgtgactggtgtgataccactaacaacaacaccaacaaaatcttattttgcaaatctcaaaggaacaaggaccagagggaaactatgcccagactgtctcaactgcacagatctggatgtggctttgggcagaccaatgtgtgtggggaccacaccttcggcgaaggcttcaatactccacgaagtcaaacctgttacatccgggtgctttcctataatgcacgacagaacaaaaatcaggcaactacccaatcttctcagaggatatgaaaatatcaggctatcaacccaaaacgtcatcgatgcggaaaaggcaccaggaggaccctacagacttggaacctcaggatcttgccctaacgctaccagtaagagcggatttttcgcaacaatggcttgggctgtcccaaaggacaacaacaaaaatgcaacgaacccactaacagtagaagtaccatacatttgtacagaaggggaagaccaaatcactgtttgggggttccattcagataacaaaacccaaatgaagaacctctatggagactcaaatcctcaaaagttcacctcatctgctaatggagtaaccacacactatgtttctcagattggcagcttcccagatcaaacagaagacggaggactaccacaaagcggcaggattgttgttgattacatgatgcaaaaacctgggaaaacaggaacaattgtctaccaaagaggtgttttgttgcctcaaaaggtgtggtgcgcgagtggcaggagcaaagtaataaaagggtccttgcctttaattggtgaagcagattgccttcatgaaaaatacggtggattaaacaaaagcaagccttactacacaggagaacatgcaaaagccataggaaattgcccaatatgggtgaaaacacctttgaagctcgccaatggaaccaaatatagacctcctgcaaaactattaaaggaaaggggtttcttcggagctattgctggtttcctagaaggaggatgggaaggaatgattgcaggctggcacggatacacatctcacggagcacatggagtggcagtggcggcggaccttaagagtacgcaagaagctataaacaagataacaaaaaatctcaattctttgagtgagctagaagtaaagaatcttcaaagactaagtggtgccatggatgaactccacaacgaaatactcgagctggatgagaaagtggatgatctcagagctgacactataagctcgcaaatagaacttgcagtcttgctttccaacgaaggaataataaacagtgaagatgagcatctattggcacttgagagaaaactaaagaaaatgctgggtccctctgctgtagagataggaaatggatgcttcgaaaccaaacacaagtgcaaccagacctgcttagacaggatagctgctggcacctttaatgcaggagaattttctctccccacttttgattcactgaacattactgctgcatctttaaatgatgatggattggataaccatactatactgctctattactcaactgctgcttctagtttggctgtaacattgatgctagctatttttattgtttatatggtctccagagacaacgtttcatgctccatctgtctataaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagattctagagtctcaagcttcggcgcgcc

SEQ ID NO: 5 - DNA, 52 bp, Artificial Sequence
  Synthesized PCR fragment containing BeYDV LIR amplified using MfeI-MluI-AscI-LIR.c
caattgacgcgtggcgcgccctagcagaaggcatgttgttgtgactccgagg

SEQ ID NO: 6 - DNA, 46 bp, Artificial Sequence
  Synthesized PCR fragment containing BeYDV LIR amplified using SbfI-AflII-HindIII-LIR.r
cctgcaggcttaagaagcttgtacgaataattcgtatccgacggaa

SEQ ID NO: 7 - DNA, 44 bp, Artificial Sequence
  Synthesized fragment containing BeYDV LIR and SIR sequences amplified using SacI-EcoRI-SIR.c
taccgagctcgaattccgagtgtacttcaagtcagttggaaatc

SEQ ID NO: 8 - DNA, 48 bp, Artificial Sequence
  Synthesized fragment containing BeYDV LIR and SIR sequences amplified using SpeI-FseI-LIR.r
actagtggccggccgtacgaataattcgtatccgacggaaatacctga

SEQ ID NO: 9 - DNA, 1303 bp, Artificial Sequence
  Synthesized annotated LIR-MCS-SIR+LIR
ctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgataaagcgtatattgtatcaggtatttccgtcggatacgaattattcgtacaagcttcttaagcctgcaggtcgactctagaggatccccgggtaccgagctcgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattccaaagaattatagaagggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtac

SEQ ID NO: 10 - DNA, 5069 bp, Artificial Sequence
  Synthesized construct No. 853
ggcgcgccctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgataaagcgtatattgtatcaggtatttccgtcggatacgaattattcgtacaagcttcttaagcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccatggcgaaaaacgttgcgattttcggcttattgttttctcttcttgtgttggttccttctcagatcttcgctgatcgaatctgcactggaataacatcttcaaactcacctcatgtggtcaaaacagccactcaaggggaggtcaatgtgactggtgtgataccactaacaacaacaccaacaaaatcttattttgcaaatctcaaaggaacaaggaccagagggaaactatgcccagactgtctcaactgcacagatctggatgtggctttgggcagaccaatgtgtgtggggaccacaccttcggcgaaggcttcaatactccacgaagtcaaacctgttacatccgggtgctttcctataatgcacgacagaacaaaaatcaggcaactacccaatcttctcagaggatatgaaaatatcaggctatcaacccaaaacgtcatcgatgcggaaaaggcaccaggaggaccctacagacttggaacctcaggatcttgccctaacgctaccagtaagagcggatttttcgcaacaatggcttgggctgtcccaaaggacaacaacaaaaatgcaacgaacccactaacagtagaagtaccatacatttgtacagaaggggaagaccaaatcactgtttgggggttccattcagataacaaaacccaaatgaagaacctctatggagactcaaatcctcaaaagttcacctcatctgctaatggagtaaccacacactatgtttctcagattggcagcttcccagatcaaacagaagacggaggactaccacaaagcggcaggattgttgttgattacatgatgcaaaaacctgggaaaacaggaacaattgtctaccaaagaggtgttttgttgcctcaaaaggtgtggtgcgcgagtggcaggagcaaagtaataaaagggtccttgcctttaattggtgaagcagattgccttcatgaaaaatacggtggattaaacaaaagcaagccttactacacaggagaacatgcaaaagccataggaaattgcccaatatgggtgaaaacacctttgaagctcgccaatggaaccaaatatagacctcctgcaaaactattaaaggaaaggggtttcttcggagctattgctggtttcctagaaggaggatgggaaggaatgattgcaggctggcacggatacacatctcacggagcacatggagtggcagtggcggcggaccttaagagtacgcaagaagctataaacaagataacaaaaaatctcaattctttgagtgagctagaagtaaagaatcttcaaagactaagtggtgccatggatgaactccacaacgaaatactcgagctggatgagaaagtggatgatctcagagctgacactataagctcgcaaatagaacttgcagtcttgctttccaacgaaggaataataaacagtgaagatgagcatctattggcacttgagagaaaactaaagaaaatgctgggtccctctgctgtagagataggaaatggatgcttcgaaaccaaacacaagtgcaaccagacctgcttagacaggatagctgctggcacctttaatgcaggagaattttctctccccacttttgattcactgaacattactgctgcatctttaaatgatgatggattggataaccatactatactgctctattactcaactgctgcttctagtttggctgtaacattgatgctagctatttttattgtttatatggtctccagagacaacgtttcatgctccatctgtctataaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagaggatccccgggtaccgagctcgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattccaaagaattatagaagggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggccactagcgcgtcaattgagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaac

SEQ ID NO: 11 - DNA, 5009 bp, Artificial Sequence
  Synthesized construct number 561
ggcgcgccctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgataaagcgtatattgtatcaggtatttccgtcggatacgaattattcgtacaagcttcttaagcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccatggcgaaaaacgttgcgattttcggcttattgttttctcttcttgtgttggttccttctcagatcttcgctgacacattatgtataggttatcatgcgaacaattcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacacctagttcagacaatggaacgtgttacccaggagatttcatcgattatgaggagctaagagagcaattaagctcagtgtcatcatttgaaaggtttgagatattccccaagacaagttcatggcccaatcatgactcgaacaaaggtgtaacggcagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccatctactagtgctgaccaacaaagtctctatcagaatgcagatacatatgtttttgtggggtcatcaagatacagcaagaagttcaagccggaaatagcaataagacccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcaaacacccaagggtgctataaacaccagcctcccatttcagaatatacatccgatcacaattggaaaatgtccaaaatatgtaaaaagcacaaaattgagactggccacaggattgaggaatatcccgtctattcaatctagaggactatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaattctgttattgaaaagatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaatgtgaagaacttatatgaaaaggtaagaagccagctaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaactgtcgccagttcattggtactggtagtctccctgggggcaatcagtttctggatgtgctctaatgggtctctacagtgtagaatatgtatttaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagaggatccccgggtaccgagctcgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattccaaagaattatagaagggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggccactagcgcgtcaattgagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaac

SEQ ID NO: 12 - DNA, 5012 bp, Artificial Sequence
  Synthesized construct number 851
ggcgcgccctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgataaagcgtatattgtatcaggtatttccgtcggatacgaattattcgtacaagcttcttaagcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccatggcgaaaaacgttgcgattttcggcttattgttttctcttcttgtgttggttccttctcagatcttcgctcaaaaacttcccggaaatgacaacagcacggcaacgctgtgccttgggcaccatgcagtaccaaacggaacgatagtgaaaacaatcacgaatgaccaaattgaagttactaatgctactgagctggttcagagttcctcaacaggtgaaatatgcgacagtcctcatcagatccttgatggagaaaactgcacactaatagatgctctattgggagaccctcagtgtgatggcttccaaaataagaaatgggacctttttgttgaacgcagcaaagcctacagcaactgttacccttatgatgtgccggattatgcctcccttaggtcactagttgcctcatccggcacactggagtttaacaatgaaagtttcaattggactggagtcactcaaaacggaacaagctctgcttgcataaggagatctaataacagtttctttagtagattgaattggttgacccacttaaaattcaaatacccagcattgaacgtgactatgccaaacaatgaaaaatttgacaaattgtacatttggggggttcaccacccgggtacggacaatgaccaaatcttcctgtatgctcaagcatcaggaagaatcacagtctctaccaaaagaagccaacaaactgtaatcccgaatatcggatctagacccagagtaaggaatatccccagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaattctgaatgcatcactccaaacggaagcattcccaatgacaaaccattccaaaatgtaaacaggatcacatacggggcctgtcccagatatgttaagcaaaacactctgaaattggcaacagggatgcgaaatgtaccagagaaacaaactagaggcatatttggcgcaatcgcgggtttcatagaaaatggttgggagggaatggtggatggttggtatggtttcaggcatcaaaattctgagggaataggacaagcagcagatctcaaaagcactcaagcagcaatcgatcaaatcaatgggaagctgaataggttgatcgggaaaaccaacgagaaattccatcagattgaaaaagagttctcagaagtcgaagggagaatccaggaccttgagaaatatgttgaggacaccaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagagatgaagcattaaacaaccggttccagatcaagggcgttgagctgaagtcaggatacaaagattggatactatggatttcctttgccatatcatgttttttgctttgtgttgctttgttggggttcatcatgtgggcctgccaaaaaggcaacattaggtgcaacatttgcatttgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagaggatccccgggtaccgagctcgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattccaaagaattatagaagggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggccactagcgcgtcaattgagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaac

SEQ ID NO: 13 - DNA, 4994 bp, Artificial Sequence
  Synthesized construct No. 852
ggcgcgccctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgataaagcgtatattgtatcaggtatttccgtcggatacgaattattcgtacaagcttcttaagcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccatggagaaaatagtgcttcttcttgcaatagtcagtcttgttaaaagtgatcagatttgcattggttaccatgcaaacaattcaacagagcaggttgacacaatcatggaaaagaacgttactgttacacatgcccaagacatactggaaaagacacacaacgggaagctctgcgatctagatggagtgaagcctctaattttaagagattgtagtgtagctggatggctcctcgggaacccaatgtgtgacgaattcatcaatgtaccggaatggtcttacatagtggagaaggccaatccaaccaatgacctctgttacccagggagtttcaacgactatgaagaactgaaacacctattgagcagaataaaccattttgagaaaattcaaatcatccccaaaagttcttggtccgatcatgaagcctcatcaggagttagctcagcatgtccatacctgggaagtccctccttttttagaaatgtggtatggcttatcaaaaagaacagtacatacccaacaataaagaaaagctacaataataccaaccaagaggatcttttggtactgtggggaattcaccatcctaatgatgcggcagagcagacaaggctatatcaaaacccaaccacctatatttccattgggacatcaacactaaaccagagattggtaccaaaaatagctactagatccaaagtaaacgggcaaagtggaaggatggagttcttctggacaattttaaaacctaatgatgcaatcaacttcgagagtaatggaaatttcattgctccagaatatgcatacaaaattgtcaagaaaggggactcagcaattatgaaaagtgaattggaatatggtaactgcaacaccaagtgtcaaactccaatgggggcgataaactctagtatgccattccacaacatacaccctctcaccatcggggaatgccccaaatatgtgaaatcaaacagattagtccttgcaacagggctcagaaatagccctcaaagagagagcagaagaaaaaagagaggactatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcaatcattgacaaaatgaacactcagtttgaggccgttggaagggaatttaataacttagaaaggagaatagagaatttaaacaagaagatggaagacgggtttctagatgtctggacttataatgccgaacttctggttctcatggaaaatgagagaactctagactttcatgactcaaatgttaagaacctctacgacaaggtccgactacagcttagggataatgcaaaggagctgggtaacggttgtttcgagttctatcacaaatgtgataatgaatgtatggaaagtataagaaacggaacgtacaactatccgcagtattcagaagaagcaagattaaaaagagaggaaataagtggggtaaaattggaatcaataggaacttaccaaatactgtcaatttattcaacagtggcgagttccctagcactggcaatcatgatggctggtctatctttatggatgtgctccaatggatcgttacaatgcagaatttgcatttaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagaggatccccgggtaccgagctcgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattccaaagaattatagaagggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggccactagcgcgtcaattgagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaac

SEQ ID NO: 14 - DNA, 2030 bp, Artificial Sequence
  Synthesized construct number 848
ctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgataaagcgtatattgtatcaggtatttccgtcggatacgaattattcgtacaagcttcttaagcctgcaggtcgactctagaggatccccgggtaccgagctcgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaattcttctttgatcatgtcaagatactcctccttagacgttgcagtctggataatagttcgccatcgtgcgtcagatttgcgaggagagaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtac

SEQ ID NO: 15 - DNA, 5800 bp, Artificial Sequence
  Synthesized construct number 475
ggcgcgccctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgataaagcgtatattgtatcaggtatttccgtcggatacgaattattcgtacaagcttcttaagcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccatggcgaaaaacgttgcgattttcggcttattgttttctcttcttgtgttggttccttctcagatcttcgctgatcgaatctgcactggaataacatcttcaaactcacctcatgtggtcaaaacagccactcaaggggaggtcaatgtgactggtgtgataccactaacaacaacaccaacaaaatcttattttgcaaatctcaaaggaacaaggaccagagggaaactatgcccagactgtctcaactgcacagatctggatgtggctttgggcagaccaatgtgtgtggggaccacaccttcggcgaaggcttcaatactccacgaagtcaaacctgttacatccgggtgctttcctataatgcacgacagaacaaaaatcaggcaactacccaatcttctcagaggatatgaaaatatcaggctatcaacccaaaacgtcatcgatgcggaaaaggcaccaggaggaccctacagacttggaacctcaggatcttgccctaacgctaccagtaagagcggatttttcgcaacaatggcttgggctgtcccaaaggacaacaacaaaaatgcaacgaacccactaacagtagaagtaccatacatttgtacagaaggggaagaccaaatcactgtttgggggttccattcagataacaaaacccaaatgaagaacctctatggagactcaaatcctcaaaagttcacctcatctgctaatggagtaaccacacactatgtttctcagattggcagcttcccagatcaaacagaagacggaggactaccacaaagcggcaggattgttgttgattacatgatgcaaaaacctgggaaaacaggaacaattgtctaccaaagaggtgttttgttgcctcaaaaggtgtggtgcgcgagtggcaggagcaaagtaataaaagggtccttgcctttaattggtgaagcagattgccttcatgaaaaatacggtggattaaacaaaagcaagccttactacacaggagaacatgcaaaagccataggaaattgcccaatatgggtgaaaacacctttgaagctcgccaatggaaccaaatatagacctcctgcaaaactattaaaggaaaggggtttcttcggagctattgctggtttcctagaaggaggatgggaaggaatgattgcaggctggcacggatacacatctcacggagcacatggagtggcagtggcggcggaccttaagagtacgcaagaagctataaacaagataacaaaaaatctcaattctttgagtgagctagaagtaaagaatcttcaaagactaagtggtgccatggatgaactccacaacgaaatactcgagctggatgagaaagtggatgatctcagagctgacactataagctcgcaaatagaacttgcagtcttgctttccaacgaaggaataataaacagtgaagatgagcatctattggcacttgagagaaaactaaagaaaatgctgggtccctctgctgtagagataggaaatggatgcttcgaaaccaaacacaagtgcaaccagacctgcttagacaggatagctgctggcacctttaatgcaggagaattttctctccccacttttgattcactgaacattactgctgcatctttaaatgatgatggattggataaccatactatactgctctattactcaactgctgcttctagtttggctgtaacattgatgctagctatttttattgtttatatggtctccagagacaacgtttcatgctccatctgtctataaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcggggtaccgagctcgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaattcttctttgatcatgtcaagatactcctccttagacgttgcagtctggataatagttcgccatcgtgcgtcagatttgcgaggagagaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggccactagtaattgagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaac

SEQ ID NO: 16 - DNA, 5740 bp, Artificial Sequence
  Synthesized construct number 471
ggcgcgccctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgataaagcgtatattgtatcaggtatttccgtcggatacgaattattcgtacaagcttcttaagcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccatggcgaaaaacgttgcgattttcggcttattgttttctcttcttgtgttggttccttctcagatcttcgctgacacattatgtataggttatcatgcgaacaattcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacacctagttcagacaatggaacgtgttacccaggagatttcatcgattatgaggagctaagagagcaattaagctcagtgtcatcatttgaaaggtttgagatattccccaagacaagttcatggcccaatcatgactcgaacaaaggtgtaacggcagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccatctactagtgctgaccaacaaagtctctatcagaatgcagatacatatgtttttgtggggtcatcaagatacagcaagaagttcaagccggaaatagcaataagacccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcaaacacccaagggtgctataaacaccagcctcccatttcagaatatacatccgatcacaattggaaaatgtccaaaatatgtaaaaagcacaaaattgagactggccacaggattgaggaatatcccgtctattcaatctagaggactatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaattctgttattgaaaagatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaatgtgaagaacttatatgaaaaggtaagaagccagctaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaactgtcgccagttcattggtactggtagtctccctgggggcaatcagtttctggatgtgctctaatgggtctctacagtgtagaatatgtatttaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcggggtaccgagctcgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaattcttctttgatcatgtcaagatactcctccttagacgttgcagtctggataatagttcgccatcgtgcgtcagatttgcgaggagagaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggccactagtaattgagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaac

SEQ ID NO: 17 - DNA, 5743 bp, Artificial Sequence
  Synthesized construct number 473
ggcgcgccctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgataaagcgtatattgtatcaggtatttccgtcggatacgaattattcgtacaagcttcttaagcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccatggcgaaaaacgttgcgattttcggcttattgttttctcttcttgtgttggttccttctcagatcttcgctcaaaaacttcccggaaatgacaacagcacggcaacgctgtgccttgggcaccatgcagtaccaaacggaacgatagtgaaaacaatcacgaatgaccaaattgaagttactaatgctactgagctggttcagagttcctcaacaggtgaaatatgcgacagtcctcatcagatccttgatggagaaaactgcacactaatagatgctctattgggagaccctcagtgtgatggcttccaaaataagaaatgggacctttttgttgaacgcagcaaagcctacagcaactgttacccttatgatgtgccggattatgcctcccttaggtcactagttgcctcatccggcacactggagtttaacaatgaaagtttcaattggactggagtcactcaaaacggaacaagctctgcttgcataaggagatctaataacagtttctttagtagattgaattggttgacccacttaaaattcaaatacccagcattgaacgtgactatgccaaacaatgaaaaatttgacaaattgtacatttggggggttcaccacccgggtacggacaatgaccaaatcttcctgtatgctcaagcatcaggaagaatcacagtctctaccaaaagaagccaacaaactgtaatcccgaatatcggatctagacccagagtaaggaatatccccagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaattctgaatgcatcactccaaacggaagcattcccaatgacaaaccattccaaaatgtaaacaggatcacatacggggcctgtcccagatatgttaagcaaaacactctgaaattggcaacagggatgcgaaatgtaccagagaaacaaactagaggcatatttggcgcaatcgcgggtttcatagaaaatggttgggagggaatggtggatggttggtatggtttcaggcatcaaaattctgagggaataggacaagcagcagatctcaaaagcactcaagcagcaatcgatcaaatcaatgggaagctgaataggttgatcgggaaaaccaacgagaaattccatcagattgaaaaagagttctcagaagtcgaagggagaatccaggaccttgagaaatatgttgaggacaccaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagagatgaagcattaaacaaccggttccagatcaagggcgttgagctgaagtcaggatacaaagattggatactatggatttcctttgccatatcatgttttttgctttgtgttgctttgttggggttcatcatgtgggcctgccaaaaaggcaacattaggtgcaacatttgcatttgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcggggtaccgagctcgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaattcttctttgatcatgtcaagatactcctccttagacgttgcagtctggataatagttcgccatcgtgcgtcagatttgcgaggagagaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggccactagtaattgagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaac

SEQ ID NO: 18 - DNA, 5725 bp, Artificial Sequence
  Synthesized construct number 474
ggcgcgccctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgataaagcgtatattgtatcaggtatttccgtcggatacgaattattcgtacaagcttcttaagcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccatggagaaaatagtgcttcttcttgcaatagtcagtcttgttaaaagtgatcagatttgcattggttaccatgcaaacaattcaacagagcaggttgacacaatcatggaaaagaacgttactgttacacatgcccaagacatactggaaaagacacacaacgggaagctctgcgatctagatggagtgaagcctctaattttaagagattgtagtgtagctggatggctcctcgggaacccaatgtgtgacgaattcatcaatgtaccggaatggtcttacatagtggagaaggccaatccaaccaatgacctctgttacccagggagtttcaacgactatgaagaactgaaacacctattgagcagaataaaccattttgagaaaattcaaatcatccccaaaagttcttggtccgatcatgaagcctcatcaggagttagctcagcatgtccatacctgggaagtccctccttttttagaaatgtggtatggcttatcaaaaagaacagtacatacccaacaataaagaaaagctacaataataccaaccaagaggatcttttggtactgtggggaattcaccatcctaatgatgcggcagagcagacaaggctatatcaaaacccaaccacctatatttccattgggacatcaacactaaaccagagattggtaccaaaaatagctactagatccaaagtaaacgggcaaagtggaaggatggagttcttctggacaattttaaaacctaatgatgcaatcaacttcgagagtaatggaaatttcattgctccagaatatgcatacaaaattgtcaagaaaggggactcagcaattatgaaaagtgaattggaatatggtaactgcaacaccaagtgtcaaactccaatgggggcgataaactctagtatgccattccacaacatacaccctctcaccatcggggaatgccccaaatatgtgaaatcaaacagattagtccttgcaacagggctcagaaatagccctcaaagagagagcagaagaaaaaagagaggactatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcaatcattgacaaaatgaacactcagtttgaggccgttggaagggaatttaataacttagaaaggagaatagagaatttaaacaagaagatggaagacgggtttctagatgtctggacttataatgccgaacttctggttctcatggaaaatgagagaactctagactttcatgactcaaatgttaagaacctctacgacaaggtccgactacagcttagggataatgcaaaggagctgggtaacggttgtttcgagttctatcacaaatgtgataatgaatgtatggaaagtataagaaacggaacgtacaactatccgcagtattcagaagaagcaagattaaaaagagaggaaataagtggggtaaaattggaatcaataggaacttaccaaatactgtcaatttattcaacagtggcgagttccctagcactggcaatcatgatggctggtctatctttatggatgtgctccaatggatcgttacaatgcagaatttgcatttaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcggggtaccgagctcgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaattcttctttgatcatgtcaagatactcctccttagacgttgcagtctggataatagttcgccatcgtgcgtcagatttgcgaggagagaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggccactagtaattgagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaac

SEQ ID NO: 19 - DNA, 25 bp, Artificial Sequence
  Synthesized primer Plasto-443c
gtattagtaattagaatttggtgtc

SEQ ID NO: 20 - DNA, 34 bp, Artificial Sequence
  Synthesized primer supP19-plasto.r
ccttgtatagctcgttccattttctctcaagatg

SEQ ID NO: 21 - DNA, 20 bp, Artificial sequence
  Synthesized primer supP19-1c
atggaacgagctatacaagg

SEQ ID NO: 22 - DNA, 32 bp, Artificial Sequence
  Synthesized primer SupP19-SacI.r
agtcgagctcttactcgctttctttttcgaag

SEQ ID NO: 23 - DNA, 509 bp, Cowpea Mosaic Virus
tattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttg